(19) United States
(12) Patent Application Publication
(10) Pub. No.: US 2025/0110105 A1
(43) Pub. Date: Apr. 3, 2025

(54) FLUID SYSTEM FRAMEWORK

(71) Applicant: Schlumberger Technology Corporation, Sugar Land, TX (US)

(72) Inventors: John Morrison Whyte, Cambridge (GB); Elizabeth Alice Gilchrist Jamie, Cambridge (GB); Priscilla Canizares-Martinez, Cambridge (GB)

(21) Appl. No.: 18/889,555

(22) Filed: Sep. 19, 2024

Related U.S. Application Data
(60) Provisional application No. 63/541,473, filed on Sep. 29, 2023.

(51) Int. Cl. G01N 33/28

(52) U.S. Cl. CPC G01N 33/2823

(57) ABSTRACT

A method may include receiving measurements of drilling fluid properties of drilling fluid during drilling indicative of a current drilling fluid condition; automatically determining a forecast drilling fluid condition based on the measurements using a model-based computational framework; analyzing the current drilling fluid condition and the future drilling condition; based on the analyzing, automatically generating a treatment recommendation and an associated projected drilling fluid condition based on the measurements using the model-based computational framework; responsive to implementation of the treatment recommendation, receiving additional measurements of the drilling fluid properties indicative of an actual drilling fluid condition; and, based at least in part on a comparison of projected drilling fluid condition and the actual drilling fluid condition, generating a quality indicator of the treatment recommendation.

### CROSS-REFERENCE TO RELATED APPLICATIONS

The subject disclosure claims priority from U.S. Application No. 63/541,473, filed on Sep. 29, 2023, herein incorporated by reference in its entirety.

### BACKGROUND

A reservoir may be a subsurface formation that may be characterized at least in part by its porosity and fluid permeability. As an example, a reservoir may be part of a basin such as a sedimentary basin. A basin may be a depression (e.g., caused by plate tectonic activity, subsidence, etc.) in which sediments accumulate. As an example, where hydrocarbon source rocks occur in combination with appropriate depth and duration of burial, a petroleum system may develop within a basin, which may form a reservoir that includes hydrocarbon fluids (e.g., oil, gas, etc.).

As an example, drilling operations may be performed to extend a borehole from a surface location to one or more target locations in a subsurface environment, for example, to produce reservoir fluid and/or to inject fluid. Drilling operations can involve use of drilling fluid, which may also be referred to as drilling mud or simply mud. Drilling fluid can serve various purposes in drilling operations, which can include lubrication and well control. Characteristics of drilling fluid can change during drilling operations, for example, through introduction of material (e.g., solids, formation fluid, etc.) and/or upon exposure to various physical phenomena (e.g., shear forces, heat, pressure, etc.). Additionally, optimal drilling fluid characteristics for one type of drilling operation may differ from those of another type of drilling operation. As a consequence, demands placed on drilling fluid can be dynamic and difficult to track during drilling operations, which can make decisions as to how to achieve desirable drilling fluid characteristics complex, which may be time consuming and costly when such decisions are to be made by a human or humans (e.g., fluids engineers, etc.).

### SUMMARY

A method may include receiving measurements of drilling fluid properties of drilling fluid during drilling indicative of a current drilling fluid condition; automatically determining a forecast drilling fluid condition based on the measurements using a model-based computational framework; analyzing the current drilling fluid condition and the future drilling condition; based on the analyzing, automatically generating a treatment recommendation and an associated projected drilling fluid condition based on the measurements using the model-based computational framework; responsive to implementation of the treatment recommendation, receiving additional measurements of the drilling fluid properties indicative of an actual drilling fluid condition; and, based at least in part on a comparison of projected drilling fluid condition and the actual drilling fluid condition, generating a quality indicator of the treatment recommendation. A system can include one or more processors; memory accessible to at least one of the one or more processors; processor-executable instructions stored in the memory and executable to instruct the system to: receive measurements of drilling fluid properties of drilling fluid during drilling indicative of a current drilling fluid condition; automatically determine a forecast drilling fluid condition based on the measurements using a model-based computational framework; analyze the current drilling fluid condition and the future drilling condition; based on the analysis, automatically generate a treatment recommendation and an associated projected drilling fluid condition based on the measurements using the model-based computational framework; responsive to implementation of the treatment recommendation, receive additional measurements of the drilling fluid properties indicative of an actual drilling fluid condition; and, based at least in part on a comparison of projected drilling fluid condition and the actual drilling fluid condition, generate a quality indicator of the treatment recommendation. One or more computer-readable storage media can include processor-executable instructions to instruct a computing system to: receive measurements of drilling fluid properties of drilling fluid during drilling indicative of a current drilling fluid condition; automatically determine a forecast drilling fluid condition based on the measurements using a model-based computational framework; analyze the current drilling fluid condition and the future drilling condition; based on the analysis, automatically generate a treatment recommendation and an associated projected drilling fluid condition based on the measurements using the model-based computational framework; responsive to implementation of the treatment recommendation, receive additional measurements of the drilling fluid properties indicative of an actual drilling fluid condition; and, based at least in part on a comparison of projected drilling fluid condition and the actual drilling fluid condition, generate a quality indicator of the treatment recommendation. Various other apparatuses, systems, methods, etc., are also disclosed.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

The following detailed description refers to the accompanying drawings. Wherever convenient Features and advantages of the described implementations may be more readily understood by reference to the following description taken in conjunction with the accompanying drawings.

FIG. 1 shows an example of a system;

FIG. 2 shows an example of a system;

FIG. 3 shows an example of a system;

FIG. 4 shows an example of a system;

FIG. 5 shows an example of a method;

FIG. 6 shows an example of a graphical user interface;

FIG. 7 shows an example of a graphical user interface;

FIG. 8 shows an example of a graphical user interface;

FIG. 9 shows an example of a graphical user interface;

FIG. 10 shows an example of a graphical user interface;

FIG. 11 shows an example of a method;

FIG. 12 shows an example of a graphical user interface;

FIG. 13 shows an example of a graphical user interface;

FIG. 14 shows an example of a graphical user interface;

FIG. 15 shows an example of a graphical user interface;

FIG. 16 shows an example of a graphical user interface;

FIG. 17 shows an example of a graphical user interface;

FIG. 18 shows an example of a graphical user interface;

FIG. 19 shows an example of a method;

FIG. 20 shows an example of a method;

FIG. 21 shows an example of a graphical user interface;

FIG. 22 shows an example of a graphical user interface;

FIG. 23 shows an example of a graphical user interface;

FIG. 24 shows an example of a graphical user interface;

FIG. 25 shows an example of a graphical user interface;

FIG. 26 shows examples of condition indexes;

FIG. 27 shows examples of condition indexes;

FIG. 28 shows an example of a method and an example of a system; and

FIG. 29 shows an example of a system.

### DETAILED DESCRIPTION

This description is not to be taken in a limiting sense, but rather is made merely for the purpose of describing the general principles of the implementations. The scope of the described implementations should be ascertained with reference to the issued claims.

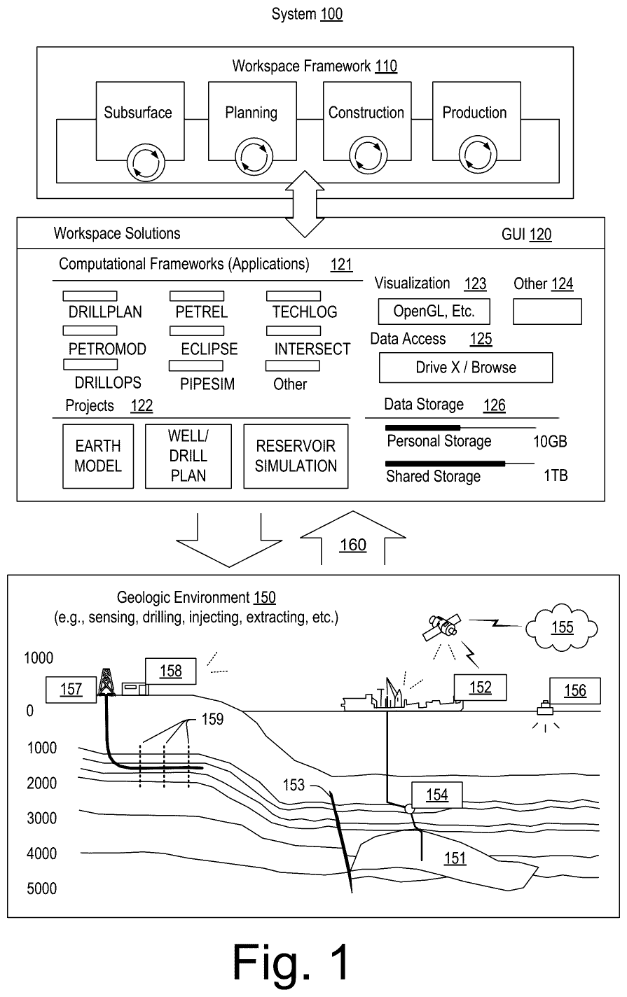

FIG. 1 shows an example of a system 100 that includes a workspace framework 110 that may provide for instantiation of, rendering of, interactions with, etc., a graphical user interface (GUI) 120. In the example of FIG. 1, the GUI 120 may include graphical controls for computational frameworks (e.g., applications, etc.) 121, projects 122, visualization features 123, one or more other features 124, data access 125, and data storage 126.

In the example of FIG. 1, the workspace framework 110 may be tailored to a particular geologic environment such as an example geologic environment 150. For example, the geologic environment 150 may include layers (e.g., stratification) that include a reservoir 151 and that may be intersected by a fault 153. As an example, the geologic environment 150 may be outfitted with a variety of sensors, detectors, actuators, etc. For example, equipment 152 may include communication circuitry to receive and to transmit information with respect to one or more networks 155. Such information may include information associated with downhole equipment 154, which may be equipment to acquire information, to assist with resource recovery, etc. Other equipment 156 may be located remote from a wellsite and include sensing, detecting, emitting or other circuitry. Such equipment may include storage and communication circuitry to store and to communicate data, instructions, etc. As an example, one or more satellites may be provided for purposes of communications, data acquisition, etc. For example, FIG. 1 shows a satellite in communication with the network 155 that may be configured for communications, noting that the satellite may additionally or alternatively include circuitry for imagery (e.g., spatial, spectral, temporal, radiometric, etc.).

FIG. 1 also shows the geologic environment 150 as optionally including equipment 157 and 158 associated with a well that includes a substantially horizontal portion that may intersect with one or more fractures 159. For example, consider a well in a shale formation that may include natural fractures, artificial fractures (e.g., hydraulic fractures) or a combination of natural and artificial fractures. As an example, a well may be drilled for a reservoir that is laterally extensive. In such an example, lateral variations in properties, stresses, etc. may exist where an assessment of such variations may assist with planning, operations, etc. to develop a laterally extensive reservoir (e.g., via fracturing, injecting, extracting, etc.). As an example, the equipment 157 and/or 158 may include components, a system, systems, etc. for fracturing, seismic sensing, analysis of seismic data, assessment of one or more fractures, etc.

In the example of FIG. 1, the GUI 120 shows some examples of computational frameworks, including the DRILLPLAN, DRILLOPS, PETREL, TECHLOG, PETROMOD, ECLIPSE, PIPESIM, and INTERSECT frameworks (SLB, Houston, Texas).

The DRILLPLAN framework provides for digital well construction planning and includes features for automation of repetitive tasks and validation workflows, enabling improved quality drilling programs (e.g., digital drilling plans, etc.) to be produced quickly with assured coherency.

The DRILLOPS framework may execute a digital drilling plan and ensures plan adherence, while delivering goal-based automation. The DRILLOPS framework may generate activity plans automatically of individual operations, whether they are monitored and/or controlled on the rig or in town. Automation may utilize data analysis and learning systems to assist and optimize tasks, such as, for example, setting ROP to drilling a stand. A preset menu of automatable drilling tasks may be rendered, and, using data analysis and models, a plan may be executed in a manner to achieve a specified goal, where, for example, measurements may be utilized for calibration. The DRILLOPS framework provides flexibility to modify and replan activities dynamically, for example, based on a live appraisal of various factors (e.g., equipment, personnel, and supplies). Well construction activities (e.g., tripping, drilling, cementing, etc.) may be continually monitored and dynamically updated using feedback from operational activities. The DRILLOPS framework may provide for various levels of automation based on planning and/or re-planning (e.g., via the DRILLPLAN framework), feedback, etc.

The PETREL framework may be part of the DELFI environment for utilization in geosciences and geoengineering, for example, to analyze subsurface data from exploration to production of fluid from a reservoir. The DELFI cognitive exploration and production (E&P) environment (SLB, Houston, Texas), referred to herein as the DELFI environment or DELFI framework, is a secure, cognitive, cloud-based collaborative environment that integrates data and workflows with digital technologies, such as artificial intelligence and machine learning.

The PETREL framework provides components that allow for optimization of various exploration, development and production operations. The PETREL framework includes seismic to simulation software components that may output information for use in increasing reservoir performance, for example, by improving asset team productivity. Through use of such a framework, various professionals (e.g., geophysicists, geologists, and reservoir engineers) may develop collaborative workflows and integrate operations to streamline processes (e.g., with respect to one or more geologic environments, etc.). Such a framework may be considered an application (e.g., executable using one or more devices) and may be considered a data-driven application (e.g., where data is input for purposes of modeling, simulating, etc.).

The TECHLOG framework may handle and process field and laboratory data for a variety of geologic environments (e.g., deepwater exploration, shale, etc.). The TECHLOG framework may structure wellbore data for analyses, planning, etc.

The PETROMOD framework provides petroleum systems modeling capabilities that may combine one or more of seismic, well, and geological information to model the evolution of a sedimentary basin. The PETROMOD framework may predict if, and how, a reservoir has been charged with hydrocarbons, including the source and timing of hydrocarbon generation, migration routes, quantities, and hydrocarbon type in the subsurface or at surface conditions.

The ECLIPSE framework provides a reservoir simulator (e.g., as a computational framework) with numerical solutions for fast and accurate prediction of dynamic behavior for various types of reservoirs and development schemes.

The INTERSECT framework provides a high-resolution reservoir simulator for simulation of detailed geological features and quantification of uncertainties, for example, by creating accurate production scenarios and, with the integration of precise models of the surface facilities and field operations, the INTERSECT framework may produce reliable results, which may be continuously updated by real-time data exchanges (e.g., from one or more types of data acquisition equipment in the field that may acquire data during one or more types of field operations, etc.). The INTERSECT framework may provide completion configurations for complex wells where such configurations may be built in the field, may provide detailed enhanced-oil-recovery (EOR) formulations where such formulations may be implemented in the field, may analyze application of steam injection and other thermal LOR techniques for implementation in the field, advanced production controls in terms of reservoir coupling and flexible field management, and flexibility to script customized solutions for improved modeling and field management control. The INTERSECT framework, as with the other example frameworks, may be utilized as part of the DELFI environment, for example, for rapid simulation of multiple concurrent cases. For example, a workflow may utilize one or more of the DELFI environment on demand reservoir simulation features.

The aforementioned DELFI environment provides various features for workflows as to subsurface analysis, planning, construction and production, for example, as illustrated in the workspace framework 110. As shown in FIG. 1, outputs from the workspace framework 110 may be utilized for directing, controlling, etc., one or more processes in the geologic environment 150 and, feedback 160, may be received via one or more interfaces in one or more forms (e.g., acquired data as to operational conditions, equipment conditions, environment conditions, etc.).

As an example, a workflow may progress to a geology and geophysics (“G&G”) service provider, which may generate a well trajectory, which may involve execution of one or more G&G frameworks (e.g., consider the PETREL framework, etc.).

In the example of FIG. 1, the visualization features 123 may be implemented via the workspace framework 110, for example, to perform tasks as associated with one or more of subsurface regions, planning operations, constructing wells and/or surface fluid networks, and producing from a reservoir.

As an example, a visualization process may implement one or more of various features that may be suitable for one or more web applications. For example, a template may involve use of the JAVASCRIPT object notation format (JSON) and/or one or more other languages/formats. As an example, a framework may include one or more converters. For example, consider a JSON to PYTHON converter and/or a PYTHON to JSON converter. Such an approach may provide for compatibility of devices, frameworks, etc., with respect to one or more sets of instructions.

As an example, visualization features may provide for visualization of various earth models, properties, etc., in one or more dimensions. As an example, visualization features may provide for rendering of information in multiple dimensions, which may optionally include multiple resolution rendering. In such an example, information being rendered may be associated with one or more frameworks and/or one or more data stores. As an example, visualization features may include one or more control features for control of equipment, which may include, for example, field equipment that may perform one or more field operations. As an example, a workflow may utilize one or more frameworks to generate information that may be utilized to control one or more types of field equipment (e.g., drilling equipment, wireline equipment, fracturing equipment, etc.).

As to a reservoir model that may be suitable for utilization by a simulator, consider acquisition of seismic data as acquired via reflection seismology, which finds use in geophysics, for example, to estimate properties of subsurface formations. As an example, reflection seismology may provide seismic data representing waves of elastic energy (e.g., as transmitted by P-waves and S-waves, in a frequency range of approximately 1 Hz to approximately 100 Hz). Seismic data may be processed and interpreted, for example, to understand better composition, fluid content, extent and geometry of subsurface rocks. Such interpretation results may be utilized to plan, simulate, perform, etc., one or more operations for production of fluid from a reservoir (e.g., reservoir rock, etc.).

Field acquisition equipment may be utilized to acquire seismic data, which may be in the form of traces where a trace may include values organized with respect to time and/or depth (e.g., consider 1D, 2D, 3D or 4D seismic data). For example, consider acquisition equipment that acquires digital samples at a rate of one sample per approximately 4 ms. Given a speed of sound in a medium or media, a sample rate may be converted to an approximate distance. For example, the speed of sound in rock may be on the order of around 5 km per second. Thus, a sample time spacing of approximately 4 ms would correspond to a sample “depth” spacing of about 10 meters (e.g., assuming a path length from source to boundary and boundary to sensor). As an example, a trace may be about 4 seconds in duration; thus, for a sampling rate of one sample at about 4 ms intervals, such a trace would include about 1000 samples where latter acquired samples correspond to deeper reflection boundaries. If the 4 second trace duration of the foregoing example is divided by two (e.g., to account for reflection), for a vertically aligned source and sensor, a deepest boundary depth may be estimated to be about 10 km (e.g., assuming a speed of sound of about 5 km per second).

As an example, a model may be a simulated version of a geologic environment. As an example, a simulator may include features for simulating physical phenomena in a geologic environment based at least in part on a model or models. A simulator, such as a reservoir simulator, may simulate fluid flow in a geologic environment based at least in part on a model that may be generated via a framework that receives seismic data. A simulator may be a computerized system (e.g., a computing system) that may execute instructions using one or more processors to solve a system of equations that describe physical phenomena subject to various constraints. In such an example, the system of equations may be spatially defined (e.g., numerically discretized) according to a spatial model that includes layers of rock, geobodies, etc., that have corresponding positions that may be based on interpretation of seismic and/or other data. A spatial model may be a cell-based model where cells are defined by a grid (e.g., a mesh). A cell in a cell-based model may represent a physical area or volume in a geologic environment where the cell may be assigned physical properties (e.g., permeability, fluid properties, etc.) that may be germane to one or more physical phenomena (e.g., fluid volume, fluid flow, pressure, etc.). A reservoir simulation model may be a spatial model that may be cell-based.

A simulator may be utilized to simulate the exploitation of a real reservoir, for example, to examine different productions scenarios to find an optimal one before production or further production occurs. A reservoir simulator will not provide an exact replica of flow in and production from a reservoir at least in part because the description of the reservoir and the boundary conditions for the equations for flow in a porous rock are generally known with an amount of uncertainty. Certain types of physical phenomena occur at a spatial scale that may be relatively small compared to size of a field. A balance may be struck between model scale and computational resources that results in model cell sizes being of the order of meters; rather than a lesser size (e.g., a level of detail of pores). A modeling and simulation workflow for multiphase flow in porous media (e.g., reservoir rock, etc.) may include generalizing real micro-scale data from macro scale observations (e.g., seismic data and well data) and upscaling to a manageable scale and problem size. Uncertainties may exist in input data and solution procedure such that simulation results too are to some extent uncertain. A process known as history matching may involve comparing simulation results to actual field data acquired during production of fluid from a field. Information gleaned from history matching, may provide for adjustments to a model, data, etc., which may help to increase accuracy of simulation.

As an example, a simulator may utilize various types of constructs, which may be referred to as entities. Entities may include earth entities or geological objects such as wells, surfaces, reservoirs, etc. Entities may include virtual representations of actual physical entities that may be reconstructed for purposes of simulation. Entities may include entities based on data acquired via sensing, observation, etc. (e.g., consider entities based at least in part on seismic data and/or other information). As an example, an entity may be characterized by one or more properties (e.g., a geometrical pillar grid entity of an earth model may be characterized by a porosity property, etc.). Such properties may represent one or more measurements (e.g., acquired data), calculations, etc.

As an example, a simulator may utilize an object-based software framework, which may include entities based on pre-defined classes to facilitate modeling and simulation. As an example, an object class may encapsulate reusable code and associated data structures. Object classes may be used to instantiate object instances for use by a program, script, etc. For example, borehole classes may define objects for representing boreholes based on well data. A model of a basin, a reservoir, etc. may include one or more boreholes where a borehole may be, for example, for measurements, injection, production, etc. As an example, a borehole may be a wellbore of a well, which may be a completed well (e.g., for production of a resource from a reservoir, for injection of material, etc.).

While several simulators are illustrated in the example of FIG. 1, one or more other simulators may be utilized, additionally or alternatively. For example, consider the VISAGE geomechanics simulator (SLB, Houston Texas) or the PIPESIM network simulator (SLB, Houston Texas), etc. The VISAGE simulator includes finite element numerical solvers that may provide simulation results such as, for example, results as to compaction and subsidence of a geologic environment, well and completion integrity in a geologic environment, cap-rock and fault-seal integrity in a geologic environment, fracture behavior in a geologic environment, thermal recovery in a geologic environment, CO2 disposal, etc. The PIPESIM simulator includes solvers that may provide simulation results such as, for example, multiphase flow results (e.g., from a reservoir to a wellhead and beyond, etc.), flowline and surface facility performance, etc. The PIPESIM simulator may be integrated, for example, with the AVOCET production operations framework (SLB, Houston Texas). As an example, a reservoir or reservoirs may be simulated with respect to one or more enhanced recovery techniques (e.g., consider a thermal process such as steam-assisted gravity drainage (SAGD), etc.). As an example, the PIPESIM simulator may be an optimizer that may optimize one or more operational scenarios at least in part via simulation of physical phenomena. The MANGROVE simulator (SLB, Houston, Texas) provides for optimization of stimulation design (e.g., stimulation treatment operations such as hydraulic fracturing) in a reservoir-centric environment. The MANGROVE framework may combine scientific and experimental work to predict geomechanical propagation of hydraulic fractures, reactivation of natural fractures, etc., along with production forecasts within 3D reservoir models (e.g., production from a drainage area of a reservoir where fluid moves via one or more types of fractures to a well and/or from a well). The MANGROVE framework may provide results pertaining to heterogeneous interactions between hydraulic and natural fracture networks, which may assist with optimization of the number and location of fracture treatment stages (e.g., stimulation treatment(s)), for example, to increased perforation efficiency and recovery.

As an example, data may include geochemical data. For example, consider data acquired using X-ray fluorescence (XRF) technology, Fourier transform infrared spectroscopy (FTIR) technology and/or wireline geochemical technology.

As an example, one or more probes may be deployed in a bore via a wireline or wirelines. As an example, a probe may emit energy and receive energy where such energy may be analyzed to help determine mineral composition of rock surrounding a bore. As an example, nuclear magnetic resonance may be implemented (e.g., via a wireline, downhole NMR probe, etc.), for example, to acquire data as to nuclear magnetic properties of elements in a formation (e.g., hydrogen, carbon, phosphorous, etc.).

As an example, lithology scanning technology may be employed to acquire and analyze data. For example, consider the LITHO SCANNER technology (SLB, Houston, Texas). As an example, a LITHO SCANNER tool may be or include a gamma ray spectroscopy tool.

As an example, a tool may be positioned to acquire information in a portion of a borehole. Analysis of such information may reveal vugs, dissolution planes (e.g., dissolution along bedding planes), stress-related features, dip events, etc. As an example, a tool may acquire information that may help to characterize a fractured reservoir, optionally where fractures may be natural and/or artificial (e.g., hydraulic fractures). Such information may assist with completions, stimulation treatment, etc. As an example, information acquired by a tool may be analyzed using a framework such as the aforementioned TECHLOG framework.

As an example, a workflow may utilize one or more types of data for one or more processes (e.g., stratigraphic modeling, basin modeling, completion designs, drilling, production, injection, etc.). As an example, one or more tools may provide data that may be used in a workflow or workflows that may implement one or more frameworks (e.g., PETREL, TECHLOG, PETROMOD, ECLIPSE, etc.).

In the example of FIG. 1, drilling may be performed in the geologic environment 150, for example, to access the reservoir 151, which may be accessed from land or offshore. In FIG. 1, the downhole equipment 154 may be, for example, part of a bottom hole assembly (BHA). The BHA may be used to drill a well. The downhole equipment 154 may communicate information to equipment at the surface, and may receive instructions and information from the equipment at the surface. During a well construction process, a variety of operations (such as cementing, wireline evaluation, testing, etc.) may be conducted. In such embodiments, data collected by tools and sensors and used for reasons such as reservoir characterization may be collected and transmitted.

A well may include a substantially horizontal portion (e.g., lateral portion) that may intersect with one or more fractures. For example, a well in a shale formation may pass through natural fractures, artificial fractures (e.g., hydraulic fractures), or a combination thereof. Such a well may be constructed using directional drilling techniques as described herein. However, these same techniques may be used in connection with other types of directional wells (such as slant wells, S-shaped wells, deep inclined wells, and others) and are not limited to horizontal wells.

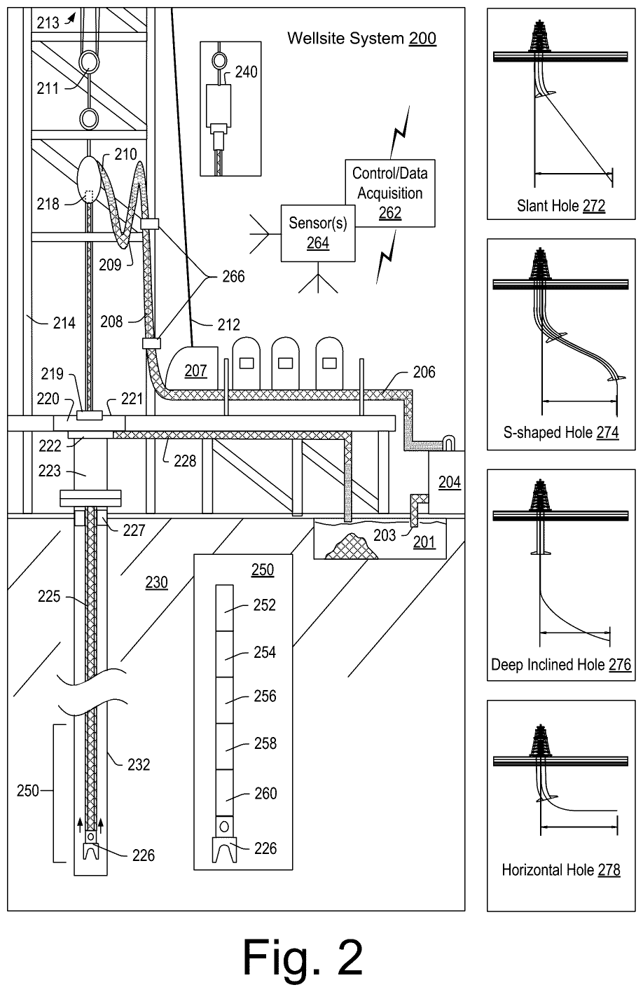

FIG. 2 shows an example of a wellsite system 200 (e.g., at a wellsite that may be onshore or offshore). As shown, the wellsite system 200 may include a mud tank 201 for holding mud and other material (e.g., where mud may be a drilling fluid), a suction line 203 that serves as an inlet to a mud pump 204 for pumping mud from the mud tank 201 such that mud flows to a vibrating hose 206, a drawworks 207 for winching drill line or drill lines 212, a standpipe 208 that receives mud from the vibrating hose 206, a kelly hose 209 that receives mud from the standpipe 208, a gooseneck or goosenecks 210, a traveling block 211, a crown block 213 for carrying the traveling block 211 via the drill line or drill lines 212, a derrick 214, a kelly 218 or a top drive 240, a kelly drive bushing 219, a rotary table 220, a drill floor 221, a bell nipple 222, one or more blowout preventors (BOPs) 223, a drillstring 225, a drill bit 226, a casing head 227 and a flow pipe 228 that carries mud and other material to, for example, the mud tank 201.

In the example system of FIG. 2, a borehole 232 is formed in subsurface formations 230 by rotary drilling; noting that various example embodiments may also use one or more directional drilling techniques, equipment, etc.

As shown in the example of FIG. 2, the drillstring 225 is suspended within the borehole 232 and has a drillstring assembly 250 that includes the drill bit 226 at its lower end. As an example, the drillstring assembly 250 may be a bottom hole assembly (BHA).

The wellsite system 200 may provide for operation of the drillstring 225 and other operations. As shown, the wellsite system 200 includes the traveling block 211 and the derrick 214 positioned over the borehole 232. As mentioned, the wellsite system 200 may include the rotary table 220 where the drillstring 225 pass through an opening in the rotary table 220.

As shown in the example of FIG. 2, the wellsite system 200 may include the kelly 218 and associated components, etc., or a top drive 240 and associated components. As to a kelly example, the kelly 218 may be a square or hexagonal metal/alloy bar with a hole drilled therein that serves as a mud flow path. The kelly 218 may be used to transmit rotary motion from the rotary table 220 via the kelly drive bushing 219 to the drillstring 225, while allowing the drillstring 225 to be lowered or raised during rotation. The kelly 218 may pass through the kelly drive bushing 219, which may be driven by the rotary table 220. As an example, the rotary table 220 may include a master bushing that operatively couples to the kelly drive bushing 219 such that rotation of the rotary table 220 may turn the kelly drive bushing 219 and hence the kelly 218. The kelly drive bushing 219 may include an inside profile matching an outside profile (e.g., square, hexagonal, etc.) of the kelly 218; however, with slightly larger dimensions so that the kelly 218 may freely move up and down inside the kelly drive bushing 219.

As to a top drive example, the top drive 240 may provide functions performed by a kelly and a rotary table. The top drive 240 may turn the drillstring 225. As an example, the top drive 240 may include one or more motors (e.g., electric and/or hydraulic) connected with appropriate gearing to a short section of pipe called a quill, that in turn may be screwed into a saver sub or the drillstring 225 itself. The top drive 240 may be suspended from the traveling block 211, so the rotary mechanism is free to travel up and down the derrick 214. As an example, a top drive 240 may allow for drilling to be performed with more joint stands than a kelly/rotary table approach.

In the example of FIG. 2, the mud tank 201 may hold mud, which may be one or more types of drilling fluids. As an example, a wellbore may be drilled to produce fluid, inject fluid or both (e.g., hydrocarbons, minerals, water, etc.).

In the example of FIG. 2, the drillstring 225 (e.g., including one or more downhole tools) may be composed of a series of pipes threadably connected together to form a long tube with the drill bit 226 at the lower end thereof. As the drillstring 225 is advanced into a wellbore for drilling, at some point in time prior to or coincident with drilling, the mud may be pumped by the pump 204 from the mud tank 201 (e.g., or other source) via the lines 206, 208 and 209 to a port of the kelly 218 or, for example, to a port of the top drive 240. The mud may then flow via a passage (e.g., or passages) in the drillstring 225 and out of ports located on the drill bit 226 (see, e.g., a directional arrow). As the mud exits the drillstring 225 via ports in the drill bit 226, it may then circulate upwardly through an annular region between an outer surface(s) of the drillstring 225 and surrounding wall(s) (e.g., open borehole, casing, etc.), as indicated by directional arrows. In such a manner, the mud lubricates the drill bit 226 and carries heat energy (e.g., frictional or other energy) and formation cuttings to the surface where the mud (e.g., and cuttings) may be returned to the mud tank 201, for example, for recirculation (e.g., with processing to remove cuttings, etc.).

The mud pumped by the pump 204 into the drillstring 225 may, after exiting the drillstring 225, form a mudcake that lines the wellbore which, among other functions, may reduce friction between the drillstring 225 and surrounding wall(s) (e.g., borehole, casing, etc.). A reduction in friction may facilitate advancing or retracting the drillstring 225. During a drilling operation, the entire drillstring 225 may be pulled from a wellbore and optionally replaced, for example, with a new or sharpened drill bit, a smaller diameter drillstring, etc. As mentioned, the act of pulling a drillstring out of a hole or replacing it in a hole is referred to as tripping. A trip may be referred to as an upward trip or an outward trip or as a downward trip or an inward trip depending on trip direction.

As an example, consider a downward trip where upon arrival of the drill bit 226 of the drillstring 225 at a bottom of a wellbore, pumping of the mud commences to lubricate the drill bit 226 for purposes of drilling to enlarge the wellbore. As mentioned, the mud may be pumped by the pump 204 into a passage of the drillstring 225 and, upon filling of the passage, the mud may be used as a transmission medium to transmit energy, for example, energy that may encode information as in mud-pulse telemetry.

As an example, mud-pulse telemetry equipment may include a downhole device configured to effect changes in pressure in the mud to create an acoustic wave or waves upon which information may be modulated. In such an example, information from downhole equipment (e.g., one or more modules of the drillstring 225) may be transmitted uphole to an uphole device, which may relay such information to other equipment for processing, control, etc.

As an example, telemetry equipment may operate via transmission of energy via the drillstring 225 itself. For example, consider a signal generator that imparts coded energy signals to the drillstring 225 and repeaters that may receive such energy and repeat it to further transmit the coded energy signals (e.g., information, etc.).

As an example, the drillstring 225 may be fitted with telemetry equipment 252 that includes a rotatable drive shaft, a turbine impeller mechanically coupled to the drive shaft such that the mud may cause the turbine impeller to rotate, a modulator rotor mechanically coupled to the drive shaft such that rotation of the turbine impeller causes said modulator rotor to rotate, a modulator stator mounted adjacent to or proximate to the modulator rotor such that rotation of the modulator rotor relative to the modulator stator creates pressure pulses in the mud, and a controllable brake for selectively braking rotation of the modulator rotor to modulate pressure pulses. In such an example, an alternator may be coupled to the aforementioned drive shaft where the alternator includes at least one stator winding electrically coupled to a control circuit to selectively short the at least one stator winding to electromagnetically brake the alternator and thereby selectively brake rotation of the modulator rotor to modulate the pressure pulses in the mud.

In the example of FIG. 2, an uphole control and/or data acquisition system 262 may include circuitry to sense pressure pulses generated by telemetry equipment 252 and, for example, communicate sensed pressure pulses or information derived therefrom for process, control, etc.

The assembly 250 of the illustrated example includes a logging-while-drilling (LWD) module 254, a measurement-while-drilling (MWD) module 256, an optional module 258, a rotary-steerable system (RSS) and/or motor 260, and the drill bit 226. Such components or modules may be referred to as tools where a drillstring may include a plurality of tools.

As to an RSS, it involves technology utilized for directional drilling. Directional drilling involves drilling into the Earth to form a deviated bore such that the trajectory of the bore is not vertical; rather, the trajectory deviates from vertical along one or more portions of the bore. As an example, consider a target that is located at a lateral distance from a surface location where a rig may be stationed. In such an example, drilling may commence with a vertical portion and then deviate from vertical such that the bore is aimed at the target and, eventually, reaches the target. Directional drilling may be implemented where a target may be inaccessible from a vertical location at the surface of the Earth, where material exists in the Earth that may impede drilling or otherwise be detrimental (e.g., consider a salt dome, etc.), where a formation is laterally extensive (e.g., consider a relatively thin yet laterally extensive reservoir), where multiple bores are to be drilled from a single surface bore, where a relief well is desired, etc.

One approach to directional drilling involves a mud motor; however, a mud motor may present some challenges depending on factors such as rate of penetration (ROP), transferring weight to a bit (e.g., weight on bit, WOB) due to friction, etc. A mud motor may be a positive displacement motor (PDM) that operates to drive a bit (e.g., during directional drilling, etc.). A PDM operates as drilling fluid is pumped through it where the PDM converts hydraulic power of the drilling fluid into mechanical power to cause the bit to rotate.

As an example, a mud motor (e.g., PDM) may be operated in different modes, which may include a rotating mode and a sliding mode. A sliding mode involves drilling with a mud motor rotating the bit downhole without rotating the drillstring from the surface. Such an operation may be conducted when a BHA has been fitted with a bent sub or a bent housing mud motor, or both, for directional drilling. Sliding may be used in building and controlling or adjusting hole angle. In directional drilling, pointing of a bit may be accomplished through a bent sub, which may have a relatively small angle offset from the axis of a drillstring, and a measurement device to determine the direction of offset. Without turning the drillstring, the bit may be rotated with mud flow through the mud motor to drill in the direction it is pointed. With steerable motors, when a desired wellbore direction is attained, the entire drillstring may be rotated to drill straight rather than at an angle. By controlling the amount of hole drilled in the sliding mode versus the rotating mode, a wellbore trajectory may be controlled rather precisely.

As an example, a PDM may operate in a combined rotating mode where surface equipment is utilized to rotate a bit of a drillstring (e.g., a rotary table, a top drive, etc.) by rotating the entire drillstring and where drilling fluid is utilized to rotate the bit of the drillstring. In such an example, a surface RPM (SRPM) may be determined by use of the surface equipment and a downhole RPM of the mud motor may be determined using various factors related to flow of drilling fluid, mud motor type, etc. As an example, in the combined rotating mode, bit RPM may be determined or estimated as a sum of the SRPM and the mud motor RPM, assuming the SRPM and the mud motor RPM are in the same direction.

As an example, a PDM mud motor may operate in a so-called sliding mode, when the drillstring is not rotated from the surface. In such an example, a bit RPM may be determined or estimated based on the RPM of the mud motor.

An RSS may drill directionally where there is continuous rotation from surface equipment, which may alleviate the sliding of a steerable motor (e.g., a PDM). An RSS may be deployed when drilling directionally (e.g., deviated, horizontal, or extended-reach wells). An RSS may aim to minimize interaction with a borehole wall, which may help to preserve borehole quality. An RSS may aim to exert a relatively consistent side force akin to stabilizers that rotate with the drillstring or orient the bit in the desired direction while continuously rotating at the same number of rotations per minute as the drillstring.

The LWD module 254 may be housed in a suitable type of drill collar and may contain one or a plurality of selected types of logging tools. It will also be understood that more than one LWD and/or MWD module may be employed. Where the position of an LWD module is mentioned, as an example, it may refer to a module at the position of the LWD module 254, the MWD module 256, etc. An LWD module may include capabilities for measuring, processing, and storing information, as well as for communicating with the surface equipment. In the illustrated example, the LWD module 254 may include a seismic measuring device.

The MWD module 256 may be housed in a suitable type of drill collar and may contain one or more devices for measuring characteristics of the drillstring 225 and the drill bit 226. As an example, the MWD module 256 may include equipment for generating electrical power, for example, to power various components of the drillstring 225. As an example, the MWD module 256 may include the telemetry equipment 252, for example, where the turbine impeller may generate power by flow of the mud; it being understood that other power and/or battery systems may be employed for purposes of powering various components. As an example, the MWD module 256 may include one or more of the following types of measuring devices: a weight-on-bit measuring device, a torque measuring device, a vibration measuring device, a shock measuring device, a stick slip measuring device, a direction measuring device, and an inclination measuring device.

FIG. 2 also shows some examples of types of holes that may be drilled. For example, consider a slant hole 272, an S-shaped hole 274, a deep inclined hole 276 and a horizontal hole 278.

As an example, a drilling operation may include directional drilling where, for example, at least a portion of a well includes a curved axis. For example, consider a radius that defines curvature where an inclination with regard to the vertical may vary until reaching an angle between about 30 degrees and about 60 degrees or, for example, an angle to about 90 degrees or possibly greater than about 90 degrees.

As an example, a directional well may include several shapes where each of the shapes may aim to meet particular operational demands. As an example, a drilling process may be performed on the basis of information as and when it is relayed to a drilling engineer. As an example, inclination and/or direction may be modified based on information received during a drilling process.

As an example, deviation of a bore may be accomplished in part by use of a downhole motor and/or a turbine. As to a motor, for example, a drillstring may include a positive displacement motor (PDM).

As an example, a system may be a steerable system and include equipment to perform methods such as geosteering. As mentioned, a steerable system may be or include an RSS. As an example, a steerable system may include a PDM or of a turbine on a lower part of a drillstring which, just above a drill bit, a bent sub may be mounted. As an example, above a PDM, MWD equipment that provides real time or near real time data of interest (e.g., inclination, direction, pressure, temperature, real weight on the drill bit, torque stress, etc.) and/or LWD equipment may be installed. As to the latter, LWD equipment may make it possible to send to the surface various types of data of interest, including for example, geological data (e.g., gamma ray log, resistivity, density and sonic logs, etc.).

The coupling of sensors providing information on the course of a well trajectory, in real time or near real time, with, for example, one or more logs characterizing the formations from a geological viewpoint, may allow for implementing a geosteering method. Such a method may include navigating a subsurface environment, for example, to follow a desired route to reach a desired target or targets.

As an example, a drillstring may include an azimuthal density neutron (ADN) tool for measuring density and porosity; a MWD tool for measuring inclination, azimuth and shocks; a compensated dual resistivity (CDR) tool for measuring resistivity and gamma ray related phenomena; one or more variable gauge stabilizers; one or more bend joints; and a geosteering tool, which may include a motor and optionally equipment for measuring and/or responding to one or more of inclination, resistivity and gamma ray related phenomena.

As an example, geosteering may include intentional directional control of a wellbore based on results of downhole geological logging measurements in a manner that aims to keep a directional wellbore within a desired region, zone (e.g., a pay zone), etc. As an example, geosteering may include directing a wellbore to keep the wellbore in a particular section of a reservoir, for example, to minimize gas and/or water breakthrough and, for example, to maximize economic production from a well that includes the wellbore.

Referring again to FIG. 2, the wellsite system 200 may include one or more sensors 264 that are operatively coupled to the control and/or data acquisition system 262. As an example, a sensor or sensors may be at surface locations. As an example, a sensor or sensors may be at downhole locations. As an example, a sensor or sensors may be at one or more remote locations that are not within a distance of the order of about one hundred meters from the wellsite system 200. As an example, a sensor or sensor may be at an offset wellsite where the wellsite system 200 and the offset wellsite are in a common field (e.g., oil and/or gas field).

As an example, one or more of the sensors 264 may be provided for tracking pipe, tracking movement of at least a portion of a drillstring, etc.

As an example, the system 200 may include one or more sensors 266 that may sense and/or transmit signals to a fluid conduit such as a drilling fluid conduit (e.g., a drilling mud conduit). For example, in the system 200, the one or more sensors 266 may be operatively coupled to portions of the standpipe 208 through which mud flows. As an example, a downhole tool may generate pulses that may travel through the mud and be sensed by one or more of the one or more sensors 266. In such an example, the downhole tool may include associated circuitry such as, for example, encoding circuitry that may encode signals, for example, to reduce demands as to transmission. As an example, circuitry at the surface may include decoding circuitry to decode encoded information transmitted at least in part via mud-pulse telemetry. As an example, circuitry at the surface may include encoder circuitry and/or decoder circuitry and circuitry downhole may include encoder circuitry and/or decoder circuitry. As an example, the system 200 may include a transmitter that may generate signals that may be transmitted downhole via mud (e.g., drilling fluid) as a transmission medium.

As an example, one or more portions of a drillstring may become stuck. The term stuck may refer to one or more of varying degrees of inability to move or remove a drillstring from a bore. As an example, in a stuck condition, it might be possible to rotate pipe or lower it back into a bore or, for example, in a stuck condition, there may be an inability to move the drillstring axially in the bore, though some amount of rotation may be possible. As an example, in a stuck condition, there may be an inability to move at least a portion of the drillstring axially and rotationally.

As to the term “stuck pipe”, this may refer to a portion of a drillstring that cannot be rotated or moved axially. As an example, a condition referred to as “differential sticking” may be a condition whereby the drillstring cannot be moved (e.g., rotated or reciprocated) along the axis of the bore. Differential sticking may occur when high-contact forces caused by low reservoir pressures, high wellbore pressures, or both, are exerted over a sufficiently large area of the drillstring. Differential sticking may have time and financial cost.

As an example, a sticking force may be a product of the differential pressure between the wellbore and the reservoir and the area that the differential pressure is acting upon. This means that a relatively low differential pressure (delta p) applied over a large working area may be just as effective in sticking pipe as may a high differential pressure applied over a small area.

As an example, a condition referred to as “mechanical sticking” may be a condition where limiting or prevention of motion of the drillstring by a mechanism other than differential pressure sticking occurs. Mechanical sticking may be caused, for example, by one or more of junk in the hole, wellbore geometry anomalies, cement, keyseats or a buildup of cuttings in the annulus.

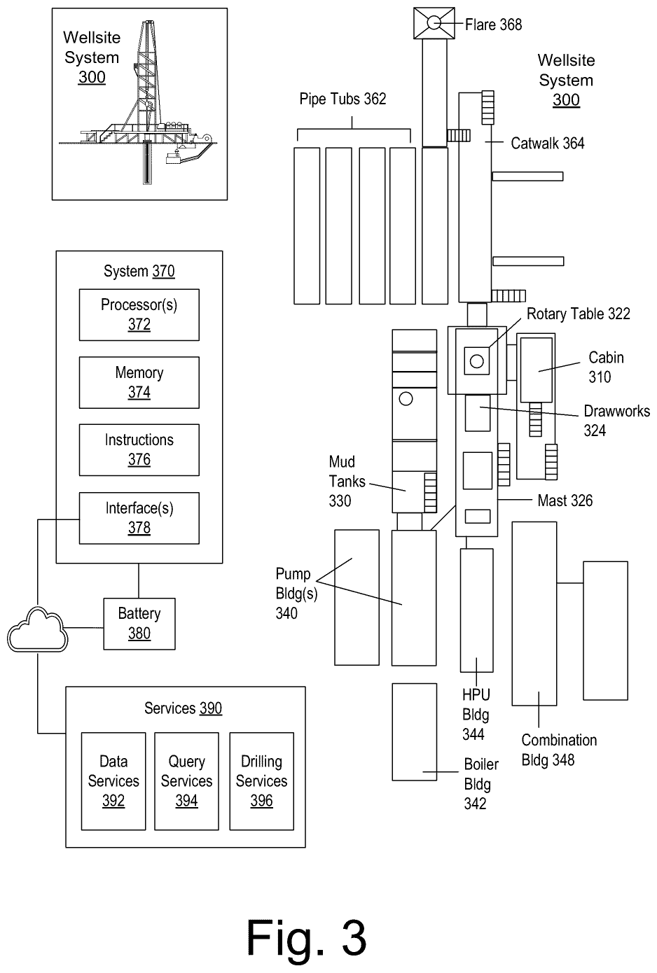

FIG. 3 shows an example of a wellsite system 300, specifically, FIG. 3 shows the wellsite system 300 in an approximate side view and an approximate plan view along with a block diagram of a system 370.

In the example of FIG. 3, the wellsite system 300 may include a cabin 310, a rotary table 322, drawworks 324, a mast 326 (e.g., optionally carrying a top drive, etc.), mud tanks 330 (e.g., with one or more pumps, one or more shakers, etc.), one or more pump buildings 340, a boiler building 342, an HPU building 344 (e.g., with a rig fuel tank, etc.), a combination building 348 (e.g., with one or more generators, etc.), pipe tubs 362, a catwalk 364, a flare 368, etc. Such equipment may include one or more associated functions and/or one or more associated operational risks, which may be risks as to time, resources, and/or humans.

As shown in the example of FIG. 3, the wellsite system 300 may include a system 370 that includes one or more processors 372, memory 374 operatively coupled to at least one of the one or more processors 372, instructions 376 that may be, for example, stored in the memory 374, and one or more interfaces 378. As an example, the system 370 may include one or more processor-readable media that include processor-executable instructions executable by at least one of the one or more processors 372 to cause the system 370 to control one or more aspects of the wellsite system 300. In such an example, the memory 374 may be or include the one or more processor-readable media where the processor-executable instructions may be or include instructions. As an example, a processor-readable medium may be a computer-readable storage medium that is not a signal and that is not a carrier wave.

FIG. 3 also shows a battery 380 that may be operatively coupled to the system 370, for example, to power the system 370. As an example, the battery 380 may be a back-up battery that operates when another power supply is unavailable for powering the system 370. As an example, the battery 380 may be operatively coupled to a network, which may be a cloud network. As an example, the battery 380 may include smart battery circuitry and may be operatively coupled to one or more pieces of equipment via a SMBus or other type of bus.

In the example of FIG. 3, services 390 are shown as being available, for example, via a cloud platform. Such services may include data services 392, query services 394 and drilling services 396. As an example, the services 390 may be part of a system such as the system 100 of FIG. 1 (e.g., consider planning services and/or operational services). As an example, the services 390 may include one or more services for directional drilling (e.g., consider a computational framework that may provide for one or more services that utilize real-time data to estimate one or more parameters, etc.).

As an example, the system 370 may be utilized to generate one or more rate of penetration drilling parameter values, which may, for example, be utilized to control one or more drilling operations.

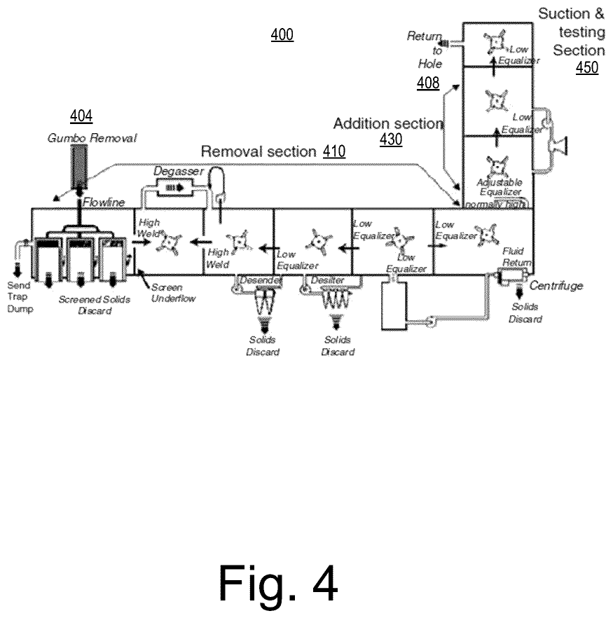

FIG. 4 shows an example of a drilling fluid (e.g., mud) system 400 that includes an inlet 404 for receiving drilling fluid from a borehole and an output 408 for flowing drilling fluid for return to a borehole. As shown, the system 400 can include a removal section 410, an addition section 430 and a suction and testing section 450. As an example, the system 400 can include a gumbo removal unit, screening units, a degassing unit, weld units, equalizer units, a centrifuge unit, etc. As shown, various units can include tanks for holding drilling fluid where one or more tanks may include one or more mixers. As an example, the addition section 430 may include one or more ports, etc., for introduction of one or more additives. As an example, a system may include a fewer number of tanks or a greater number of tanks than the system 400. As an example, tanks may include a trip tank that may be utilized during tripping operations (e.g., running into hole (RIH), pulling out of hole (POOH), etc.).

As to the term “gumbo”, it may refer to a nonspecific type of shale that becomes sticky when wet and adheres aggressively to surfaces. For example, gumbo may form mud rings and balls that can plug an annulus, a flowline and shale-shaker screens. Gumbo is likely to contain appreciable amounts of Ca+2 smectite clays and may be dispersed in a water mud, causing rapid accumulations of colloidal solids.

As an example, drilling fluid may be an oil-based mud (OBM), which can include synthetic oil-based mud (SOBM), or a water-based mud (WBM). As to OBM, it may be an invert-emulsion mud, or an emulsion whose continuous phase is oil. Various types of commercial oil muds may be formulated with 5 vol. % water or more or less than 5 vol. % water. Various types of nonwater-base drilling fluids can include synthetic fluid (e.g., ethers, esters, olefin oligomers, blends, etc.), diesel oil, and/or mineral oil (e.g., ordinary and enhanced purity). As to water-base muds (WBMs), they can be defined as drilling fluid (mud) in which water or saltwater is the major liquid phase as well as the wetting (external) phase. General categories of water-base muds include fresh water, seawater, salt water, lime, potassium and silicate. As an example, a water-base mud can be a clear water, clay base, silicate, clay and polymer, polymer with low or no clay, clear brine, etc.

Various types of muds (e.g., drilling fluids, lubricants, etc.) may take time and resources to formulate. For example, consider a recipe for making mud that commences with one or more base materials, one or more ingredients, one or more premixes, etc., where the order of ingredients, mixing times, temperatures, etc., may be controlled. A formulated mud may be of considerable value and suitable for reuse. For example, after an operation is performed, at least a portion of the mud may be collected (e.g., in a tank, etc.) and transported to another site for use.

As an example, where mud becomes contaminated beyond an acceptable level, processing and/or disposal may be options, which can add cost, time, resources, etc. As to disposal, various regulations can exist that control how and/or where mud is disposed. In general, mud with less contamination may be easier and less expensive to handle than mud with more contamination.

As an example, a framework can provide automated treatment advice for drilling fluid systems. For example, consider a framework that can include various components for making determinations as to fluid treatment, which may be part of a more expansive fluids advisor system. As an example, a framework may include components built using data sets and physical and chemical knowledge that can provide treatment recommendations based on signatures in digital fluids data. For example, consider a framework that can receive data, determine a signature and, based on the signature, generate a recommendation, which may be implemented in the field, for example, as one or more control actions.

As an example, a framework can provide qualitative and quantitative advice on fluid treatments to drilling fluids specialists at rig site and/or operating remotely and/or control instructions to equipment at a rig site. Such advice may be based on analysis of customized annotated data sets for use in signature detection in situations where a treatment may be desirable. In such an approach, one or more automated fluid monitoring systems may inform recommendations and facilitate fluids domain knowledge.

As an example, a framework may operate as a tool that can generate advice, which may be in the form of instructions for one or more levels of control (e.g., automated levels, semi-automated levels, manual levels, etc.). Such advice may be based on one or more determinations as to benefits of treating fluid of a fluid system while drilling, which may help to improve drilling. As an example, advice may be to recommend what action or actions may be taken to rectify contamination in fluid.

As an example, a framework can provide for selecting an appropriate treatment to modify a drilling fluid, for example, from a non-ideal state to a desired state with desired properties. In such an example, the framework may provide output to relatively complex scenarios where the framework is aided by knowledge of data signals that can indicate that one or more issues exist (e.g., contamination, etc.). As an example, a framework may generate a treatment decision on the basis of knowledge of chemistry and behavior of a fluid system and of one or more factors such as, for example, potential additives, formation being drilled, products available at a rig site, drilling parameters (e.g., flow rate, active system volume, bit depth, effective density, etc.), solids control system, mixing capabilities at a rig site, etc.

As to effective density (ECD), it may be defined as the effective density exerted by a circulating fluid against a formation that takes into account a pressure drop in an annulus above a point being considered. For example, ECD may be computed as: d+P/(0.052*D), where d is the mud weight (ppg), P is the pressure drop in the annulus between depth D and surface (psi), and D is the true vertical depth (feet). ECD may be a parameter utilized to assess scenarios involving kicks and losses (e.g., particularly in wells that have a narrow window between a fracture gradient and pore-pressure gradient).

While general guidelines may exist for fluid treatment, such static information will not provide for a data-driven approach for automatically identifying when a fluid may benefit from treatment nor for a data-driven approach for automatically generating instructions as to how to make and implement a treatment that may be beneficial. In various scenarios, choice of product may be relevant along with factors such as one or more of addition rate, concentration, mixing regime, etc. For example, a fluids specialist might know that a particular additive may be added to increase viscosity but not know how quickly to add that additive nor not know what desired target properties are to be achieved (e.g., in a time-dependent manner). In such an example, the fluids specialist may miss a window of opportunity during drilling operations that may introduce some amount of non-productive time (NPT) and/or that may actually alter fluid properties too late in a manner that may be detrimental or suboptimal for a subsequent field operation (e.g., POOH, etc.).

As an example, a framework may be part of a system and/or operatively coupled to a system to provide automated treatment advice on a relatively extensive range of fluid properties where the framework may learn from treatment decisions to inform future decisions, creating a self-learning fluid treatment framework.

As explained, a framework can generate fluid treatment advice, particularly as to properties of a drilling fluid. In such an example, various measurements of particular fluid properties may be checked for corresponding positions within corresponding specifications and/or target windows. For example, depending on a measurement's position and its relation to a user-defined previous set of data points, a framework may either give no advice as everything is normal, or flag a situation to a user that the framework believes a decision on treatment would be beneficial (e.g., taking no action would be a valid decision). As to being beneficial, such a decision may be beneficial for a current operation and/or a future operation (e.g., drilling, POOH, RIH, etc.) and/or to help maintain wellbore stability (e.g., diminish risk of kick, washout, etc.).

As to some examples of categories that may be utilized in detection and/or decision making, consider one or more of: property out-of-specification (OOS) (e.g., higher than maximum); property out-of-specification (OOS) (e.g., lower than minimum); rising trend (e.g., within specification limits but consecutive increases); falling trend (e.g., within specification limits but consecutive decreases); large increase (e.g., within specifications but an unexpectedly large single increase); large decrease (e.g., within specifications but an unexpectedly large single increase); accelerating increase (e.g., within specifications but with the rate of increase accelerating over a user-defined number of data points); and accelerating increase (e.g., within specifications but with the rate of increase accelerating over a user-defined number of data points).

As an example, when one of a number of conditions is met, a framework may issue a signal, for example, as a visual alert of a human machine interface (HMI) that may also provide contextual advice on how to deal with an issue. As an example, information may be provided by a fluid system-specific treatment type of encyclopedia that includes detailed treatment advice for various situations encountered. As an example, advice provided may be a mixture of qualitative advice and semi-quantitative (e.g., recommend a concentration to cause a change, such as adding 0.75 kg/m³ of a particular additive to increase yield point (YP) and non-treatment advice such as recommending that titrations are repeated to check for influxes).

As an example, generated advice may also vary depending on position of a most recent measurement(s) within one or more specification windows. For example, if a property has seen consecutive rises but is in the lower half of a specification window, the advice provided may be different than if the same situation occurs in the top half. Such an approach can provide more suitable advice as, otherwise, there may be an implication that an action that is otherwise good (e.g., a property is low and is rising due to prescribed treatment) is a problem to be mitigated as if it were close to going out of control. With similar logic, an acceleration in a change of a property may be treated by a framework as more concerning than when a slow/steady rate of change is flagged; advice on how to deal with each situation is given accordingly.

As an example, a framework may generate a mix ticket function. For example, consider a fluids advisor framework that may produce a mix ticket responsive to receipt of a signal, which may be at the request of a user, a condition, an issue, etc. In such an example, at a basic level, a framework may highlight an area of concern such that the framework provides advice on how to improve the situation. In various instances, it may be unusual for solely one property to require treatment. In such situations, an advisor framework may produce a combined treatment recommendation where multiple treatments are combined and output (e.g., to a display, as machine instructions, etc.). Such an approach may be achieved by detection of properties that demand treatment and by identifying one or more corresponding treatments. As an example, a framework may weight and/or otherwise assess importance of each treatment and then prioritize them accordingly (e.g., per a weighting of each). In such an example, a framework may include one or more customizable routines such that operation may be customizable by users and to provide for combining factors in such a way as to incorporate potential cross-property treatments.

As an example, consider a generated recommendation that calls for increasing yield point (YP) in a FLO-THRU system and reducing API fluid-loss simultaneously. In such an example, a framework may assess the relative importance of each to decide if a combined treatment is appropriate, which may be followed by identifying recommended treatments, which, in this case, may provide for adding FLO-TROL and DUO-VIS and then assessing one or more synergistic effects. In such an example, the two additives will increase YP, noting that DUO-VIS will increase it more than FLO-TROL but that only FLO-TROL will reduce API fluid-loss. In this case, the framework may take a default recommendation for each product and modify it as a result of the combination. For example, if in principle, 0.75 kg/m³ of DUO-VIS is recommended along with 0.5 kg/m³ of FLO-TROL, the framework may identify the synergistic effect on YP and recommend a reduced concentration of DUO-VIS. Such an approach may be extended to as a number of treatments.

As an example, a framework may provide for automated treatment assessment, procedural adherence and detection of one or more side-effects. For example, consider an approach that involves labelling each product with data on how it affects each fluid property and its intended function(s) (e.g., primary, secondary, etc.), such that a fluids advisor framework has knowledge of what properties may be affected upon the addition of a component to an active system. In such an example, by ingesting data on flow rate and well volume, the framework becomes aware of when effects may first be observed (e.g., via receipt of sensor data, etc.). Such an approach enables a framework to identify the beginning of a desired treatment response and to discriminate between this and an undesired response. As an example, consider the addition of DUO-VIS to a FLOPRO system. In such an example, a framework has knowledge that FLOPRO is expected to raise YP, just as it also has knowledge that the effect is expected to be seen after around 90 minutes from its addition. Given such awareness, if a significant rise in YP is detected after 20 minutes, the framework can flag this observed behavior as a potential problem because it is unlikely that the treatment, if implemented as prescribed, would have taken effect so rapidly and be circulated around a wellbore, to be measured. Assuming that there are no early warnings, in such an approach, a framework system may watch for one or more changes to an expected property and if/when the one or more changes occur. In such an approach, the framework may store the result with an assessment of its success. Such an example may be extended such as, for example, consider an increase in YP of 10 percent after two full circulations, which may be classed as a successful treatment; whereas, no chance or an increase of 3 percent would not be deemed successful.

As an example, an advisor framework can help to assess procedural adherence by measuring and storing time between advice being given to the advice being implemented (e.g., being followed) and/or time from advice being given to one or more properties attaining one or more corresponding target values. For example, consider a scenario where, if a property value falls below its specified minimum, then the advisor framework will assess if the action recommended was taken, when it was taken and how many attempts may have been taken. In such an example, the framework may provide an assessment as to hours not spent at optimum as to fluid management. In various instances, a framework may provide for a decrease in hours not spent at optimum, or, in other words, provide for an increase in hours spent at optimum.

As an example, a framework may provide automated treatment assessment capabilities that provide for identification of side-effects of treatments that, while seemingly unrelated to the treatments made, have nevertheless occurred. Such an approach may be utilized to advance self-learning of one or more features of a framework, which may provide for generation of treatment prioritizations. For example, consider an advisor framework that includes a capability to look (e.g., in relevant time periods such as, for example, one or two full circulations) at one or more channels other than those expected to be affected by a particular treatment or treatments. In such an example, ff an unrelated/untargeted property changes substantially in conjunction with a treatment designed to alter something else, the framework may record such behavior, for example, as part of a treatment data object as an observed side-effect. For example, a framework may identify that X did happen even though it was not expected to happen and that it may be a detrimental side-effect (e.g., or a neutral or beneficial side-effect).

As an example, a framework can provide for self-learning such that a self-learning treatment system may be implemented as part of a fluid system. As an example, a fluids advisor framework can include features that, provided with a sufficiently large dataset, can produce treatment recommendations that are informed by self-learning from previous pieces of advice given and treatments made. In such an example, the framework may provide for creating data objects for both treatments and advice given. In such an example, when the advisor framework gives treatment advice (e.g., to a fluid engineer, a machine, etc.), that point may be time-stamped and a data object formed. In such an example, the object may include the advice given, time ofadvice, the time ofimplementation or not (e.g., a machine or human decision upon following it (or not)), depth of a borehole, one or more properties advised upon, information pertaining to what prompted the advice, the advice given, and an official response from a machine (e.g., controller, etc.) and/or a human. As an example, an object may be defined according to one or more specifications, which may pertain to one or more types of drilling fluid, geologic regions, etc. For example, consider an object that may be defined using over 10 data channels, which may include data channels for which data are generated in context and/or after treatment (e.g., addition of one or more additives, etc.). As an example, initial data and additional data may be combined and stored as a data object for an instance of generated advice (e.g., a recommendation).

TABLE 1
Selected data from advice data object

| | | | | | Advice | |
|---|---|---|---|---|---|---|
| Decision Time | Advice Time | D | Prop. | Trig. | Given | Impl. |
| 20xx-11-12T | 20xx-11-12T | 2066 | Density | OOS | Dilute | Followed |
| 08:15:00.000Z | 07:15:00.000Z | | | High | system with | |
| | | | | | premix/ | |
| | | | | | base brine | |
| | | | | | over two | |
| | | | | | full | |
| | | | | | circulations | |
| 20xx-11-12T | 20xx-11-12T | 2066 | R6 | OOS | Dilute | Followed |
| 08:15:00.000Z | 07:15:00.000Z | | | High | system with | |
| | | | | | premix base | |
| | | | | | brine. | |

As an example, a framework can provide for creation of a treatment object in a manner akin to that of an advice object; noting that the two may not be interdependent. For example, treatments may be recorded as their own objects regardless of whether they were advised by a framework or not. As an example, a framework may be informed (e.g., via machine, human, etc.) as to start of an addition of one or more additives. Such a time may be recorded along with identity of one or more additives, along with mass being added, packaging and one or more other relevant pieces of information. As to a treatment object, it may include one or more reasons for the treatment being made and one or more assessments of success after a number or numbers of circulations (e.g., after one and two full circulations). By having this information captured in the form of a data object, a framework can provide for assessing success and/or failure of each treatment made. In various examples, information may include one or more human inputs, which may include human expert input (e.g., domain expert) such that a framework may compare a human's viewpoint to that of the framework (e.g., an automated treatment assessment that the framework is generating). Potential outcomes from such a comparison may provide for tuning of treatment assessment, for example, with a longer-term goal of making advice more quantitative, for example, to understand better what movement in a property represents a successful treatment. As an example, automated assignations to a batch or a wave of treatments may also be made by an advisor framework, which may provide for various analyses and operations of the advisor framework.

TABLE 2
Selected data from advice data object

| | | | | | | Treat. | Treat. |
|---|---|---|---|---|---|---|---|
| Current | Treat. | | | Mix | | Result @ | Result @ |
| Op. | Comp. | Unit | Actual | Loc. | Reason | 1TCT | 2TCT |
| Cond. | EDC 95-11 | bbl | 56 | Active | Inc. | Inc. in OWR | Circ. |
| System | | | | Pit | OWR | | Stopped |
| Cond. | M-I WATE | MT | 8 | Active | Inc. Fluid | Inc. in | Circ. |
| System | UFG | | | Pit | weight | Density | Stopped |

As explained, a framework can be structured in a manner that provides for self-learning. As explained, a framework may provide for generation of one or more data objects that may be stored to one or more storage devices where, for example, learning may be performed online, offline, locally and/or remotely. As to learning, one or more machine learning models may be utilized. As an example, one or more objects may be defined in a manner to facilitate learning, which may include supervised learning and/or unsupervised learning.

As an example, a learning workflow may be performed where an advisor framework detects automatically a situation that can benefit from advice. In such an example, based on an existing treatment encyclopedia, the framework may provide a recommendation and record relevant contextual information, with the response from a system (e.g., and/or humans) being recorded. As an example, a weighting may be applied to a recommendation (e.g., under corresponding circumstances) according to whether success or failure is recorded, for example, by a system and/or by one or more engineers. As an example, a subsequent re-weighting of a recommendation may be performed based on how positive an effect was observed as to a fluid's condition index. For example, a framework may determine how closely a treatment moved a fluid towards an “ideal” fluid under the circumstances. In such an example, weighting may be recorded and then fed back into one or more components of a framework, which may act to update a treatment encyclopedia. As an example, consider that in initial wells, a framework may recommend addition of DUO-VIS to increase YP of a FLOPRO system. In such an example, after a sufficient amount of self-learning, the framework might still recommend DUO-VIS but may recommend also adding a higher concentration, adding FLO-TROL in addition due to how a fluid system has reacted historically under similar conditions or, for example, the framework might inform a user and/or a machine, that under the circumstances, no treatment has sufficiently high success rates to recommend expenditure of resources (e.g., additive cost, time, machine activity, etc.).

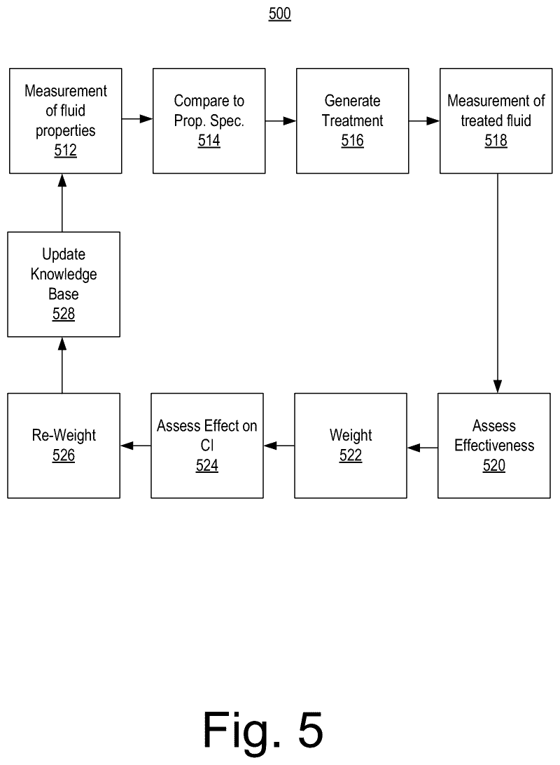

FIG. 5 shows an example of a method 500 that includes a measurement block 512 for measurement of fluid properties, a comparison block 514 for comparison of measurement of fluid properties to one or more property specifications, a generation block 516 for generation of a treatment (e.g., advice or recommendation), a measurement block 518 for measurement of treated fluid properties, an assessment block 520 for assessment of effectiveness of the treatment, a weighting block 522 for weighting one or more factors, an assessment block 524 for assessing effectiveness of the treatment, as weighted, on a condition index (CI) (e.g., for each fluid property while drilling, etc.), a re-weighting block 526 for re-weighting one or more factors, and an update block 528 for updating a knowledge base.

As to a condition index (CI), it may be defined for monitoring evolution of drilling fluids during drilling operations (e.g., drilling to extend a borehole, running in hole (RIH), pulling out of hole (POOH), etc.). As an example, a framework may operate as a comprehensive fluids advisor system using a CI. For example, custom data sets and fluid chemistry knowledge may be utilized to build a CI to assess performance of drilling fluid in real-time. In such an approach, a suitable treatment can be prescribed, and the state and performance of fluid post-treatment can be monitored.

As explained, a framework can operate to provide a holistic assessment of a drilling fluid's condition to aid drilling fluids specialists and/or controllers, at a rig site and/or operating remotely, in selecting the most suitable treatment to control or improve performance. A CI may be a tailored index that combines real-time fluid measurements and input from one or more sources (e.g., a controller, a drilling engineer, etc.). As an example, a CI can quantify performance of drilling fluid to determine when a treatment is required in an automated way.

Monitoring the state of drilling fluids tends to be a complex, time-consuming task prone to error. As an example, a framework can facilitate this task and quantify drilling fluid health. For example, such a framework can compute a condition index (CI) for each fluid property while drilling. Such CIs may be tunable by a fluid expert, for example, according to operational needs, where one or more CIs may be exploited to construct a global CI that encapsulates an overall fluid state. A framework may operate in a robust and customizable manner, making it amenable to seamless assimilation in a fluid advisor workflow.

As an example, one or more CIs can be used to assess the effects of a treatment on the entire fluid system along with individual properties. By linking a CI-based assessment to time series fluid treatment data, a framework may distinguish between the effect of a single treatment having a large effect and three treatments having smaller individual but larger overall effects (e.g., positive and/or negative). As an example, a framework can determine relative importance of each property to be incorporated into an assessment and directionality of each property. As an example, such an approach may be used to assess the relative quality of complex fluid and/or other systems, whether in the oil and gas industry and/or one or more other industries.

As an example, a framework can operate using one or more condition indexes. For example, consider a global condition index (CI) to assess fluid performance; a double-weighting element to reflect the relative importance of specific properties to the CI and their directionality; a holistic treatment assessment using one or more CIs and time series treatment data; and a self-learning process that can recommend treatments and predict likely effects.

As to a global condition index (GCI), it may be used to assess the global performance of drilling fluids. As an example, a controller and/or a fluids engineer may monitor evolution of rheology and chemical properties around a given target value or values. Due to the drilling process, properties of fluid tend to change and depart from one or more working target values. In various instances, each fluid property may have a maximum and/or a minimum, which may be above and below a respective target. Such extrema may be referred to as specification values and find use by a fluids engineer to adjust a drilling fluid formula.

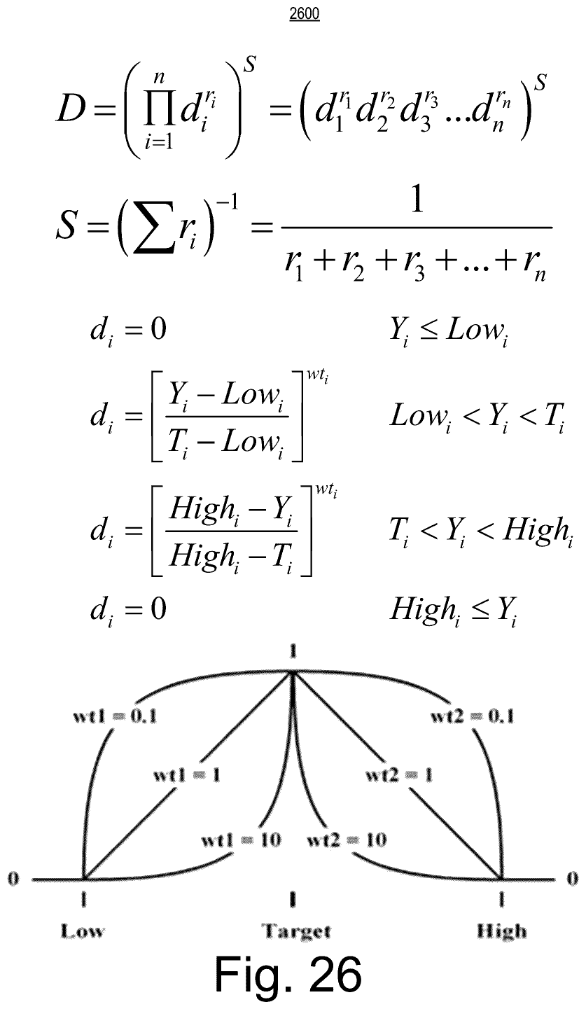

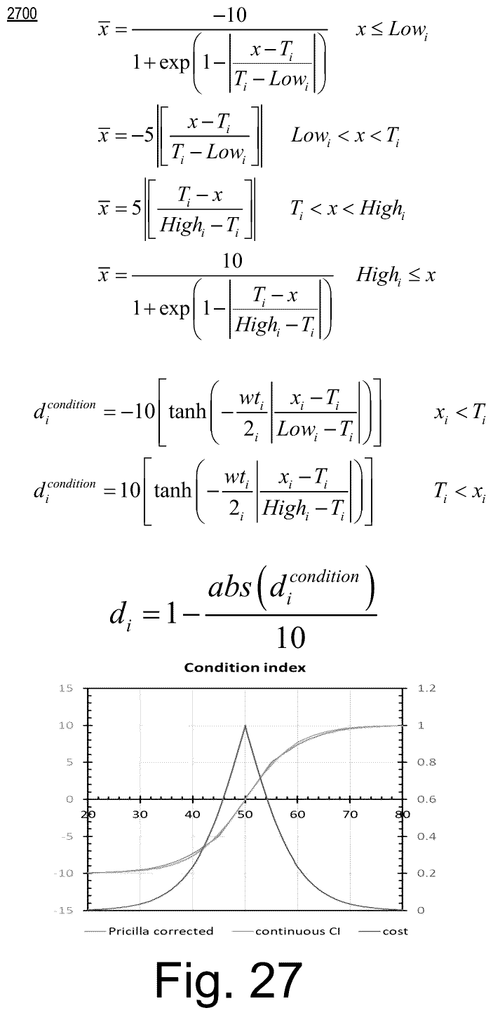

As to various examples of condition indexes, some examples are also presented in FIGS. 26 and 27; noting that one or more types of condition indexes may be formulated and utilized by a framework or frameworks.

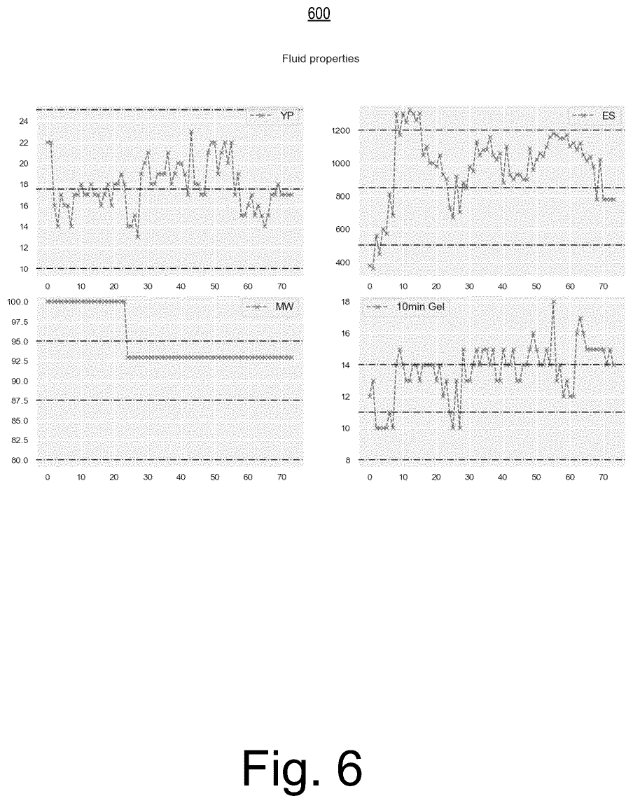

FIG. 6 shows a series of plots 600 of time series samples for yield point (YP), electric stability (ES), density (e.g., mud weight) and 10 minute gel (e.g., gel test). In these example plots 600, target values and extrema may be included. As an example, a framework may provide for generating one or more graphical user interfaces (GUIs) that can present such plots, along with target values and extrema.

As to YP, it may be defined as the elastic limit at which a material will lose its elasticity and deform permanently. For drilling fluids, yield point can refer to the resistance of initial flow of the fluid or in other words, the stress required to start the movement of the fluid. As an example, ES may increase as percentage of cuttings increase. An increase in emulsion stability of mud may indicate an amount of voltage required to break down the emulsion and allow the emulsified water droplet to connect, allowing electrical current to flow. As to density or mud weight, it is the density of the drilling fluid and may be measured in pounds per gallon (lb/gal) (ppg) or pound cubic feet (pcf). In the field it may be measured using a mud scale or mud balance. As an example, mud may weigh up to 22 ppg or 23 ppg or more; noting that a gallon of water may weigh approximately 8.33 pounds (or 7.48 ppg). As to a 10 minute gel strength test, it can indicate a maximum dial deflection as per API, for example, after the mud has remained static for 10 minutes, and can be the shear stress measured at low shear rate after the mud is allowed to set for 10 minutes. Gel strength tends to be time dependent and may be measured in a range, for example, of 10 seconds to 10 minutes. Gel strength is a rheological parameter that can help to understand drilling fluid properties for various drilling projects.

As an example, a framework can construct one or more condition indexes based on drilling-fluid measurements to assess the state of fluid while drilling. As explained, such CIs may provide for selection of the most appropriate treatment to maintain and/or improve drilling performance. As simultaneously monitoring several fluid properties can be a complex and time-consuming task prone to error, use of an automated framework can make such tasks more robust and efficient by formulating condition indexes for each of the properties of interest.

As an example, a framework may operate in a multi-stage manner, for example, using the following two states. As to a first stage, consider splitting drilling-fluid samples into four regions according to their initial specifications, and, without loss of generality, mapping these measurements into a range [−10,10]. In this way, a framework may obtain the following four regions: (i) measurements above the target value and within specification are mapped to the region [0,5], (ii) measurements above target and out of specifications are mapped to [5,10], (ii) measurements below target and within specification are mapped to the region [−5,0], and (iv) measurements below target and out of specification are mapped to the region [−10, −5]. As to a second stage, values inside specifications, (i) and (ii) can be by default bounded and transformed, for example, using linear mapping. And, values outside specifications can, in principle, take any value and be transformed, for example, using a non-linear mapping (e.g., consider a logistic function). In such an approach, rescaled measurements can be condition indexes for each of the fluid properties considered.

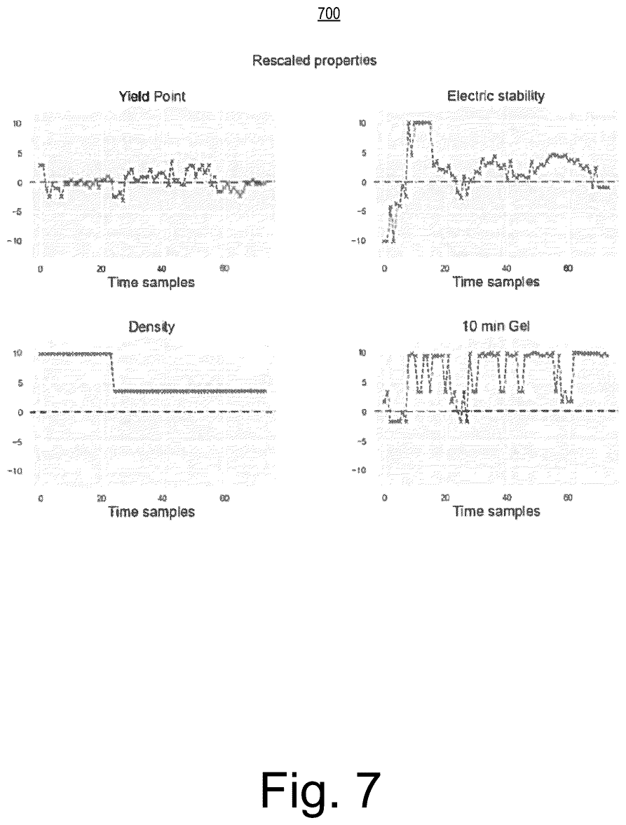

FIG. 7 shows a series of plots 700 of time series samples as rescaled for yield point (YP), electric stability (ES), density and 10 minute gel. In these example plots 700, target values and extrema may be included. As an example, a framework may provide for generating one or more graphical user interfaces (GUIs) that can present such plots, along with target values and extrema.

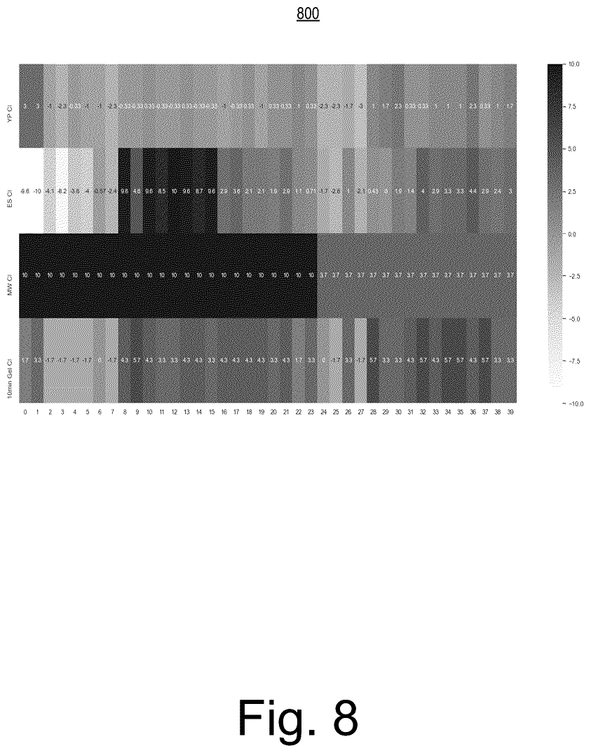

FIG. 8 shows an example of a heat map 800 of fluid properties versus time for time samples. As shown, the fluid properties can be in the form of condition indexes for properties, for example, as shown in the plots 600 and 700 of FIGS. 6 and 7.

To facilitate monitoring of condition indexes, a framework may provide for generation of one or more GUIs for visualization that exploits the fact that the condition indexes that in absolute value are a timeseries restricted to the range [0-10]. For example, a framework can map the CIs into a circle using the following transformation:

$${CI}_{n} = {\vert CI \vert}_{n} {e}^{i ω ts}$$

In the foregoing equation, n is a given fluid property, CI, is the corresponding condition index, omega is a given sampling frequency, and ts is the sample acquisition time. In such an approach, condition indexes inside specifications, can be mapped into a circle (e.g., of radius 5) and condition indexes outside specifications can be mapped into another circle (e.g., of radius of 10).

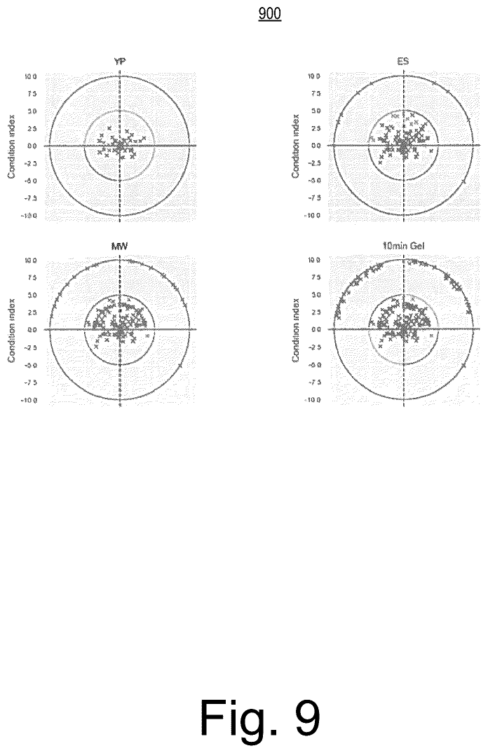

FIG. 9 shows a series of plots 900 of time series samples as transformed for yield point (YP), electric stability (ES), density and 10 minute gel. In these example plots 900, target values and extrema may be included. As an example, a framework may provide for generating one or more graphical user interfaces (GUIs) that can present such plots, along with target values and extrema. In the example plots 900, the radii are equal to the absolute value of corresponding CI values and their phase is given by the sampling time, which is set to 10 minutes.

As an example, a double weighting approach may be utilized by a framework. For example, consider an approach that includes a reweighting or tuning parameter that allows for adjustment to a relative importance of negative condition indexes according to a drilling operation. In such an example, weights can be set before the operation starts or during the drilling process. After reweighting, a global condition index may be computed as follows:

$${CI}_{n} = \frac{1}{n} {\sum }_{1}^{n} {β}_{n} \vert {CI}_{n , i} \vert$$

In the foregoing equation, n is the number of fluid properties considered, CI is the condition index, i is a given sample and bₙ is the tuning parameter defined by:

$${β}_{n} = 1 , if {CI}_{n , i} > 0$$

$${β}_{n} = 0 , if {CI}_{n , i} < 0$$

As an example, the tuning parameter may be given by a fluids engineer. As an example, by averaging the absolute value of the reweighted condition indexes, a framework can generate a unique condition index that provides a direct measure of the state of the drilling fluid. For example, the higher the condition index, the farther the fluid is from its optimal working condition.

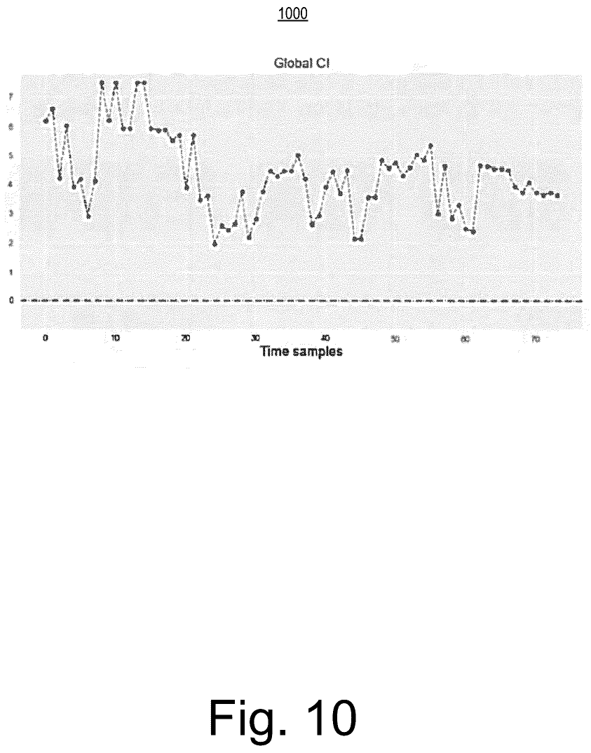

FIG. 10 shows an example of a plot 1000 of a global condition index (GCI) versus time for time samples. As explained, a higher value of GC can indicate that a fluid is further from its optimal working condition, which may be utilized as a trigger (e.g., according to a threshold, one or more rate criteria, etc.), for generation of one or more recommendations.

As an example, a framework may provide for treatment effectiveness assessment. For example, time series data for fluid treatments can become increasingly available and by combining these data with global condition index characterizations, a framework can assess holistic effectiveness of a treatment. Such an approach can address shortcomings of treatments that are made to target specific properties but can affect almost all other properties to varying degrees, which can make a manual holistic assessment quite challenging. As to how a treatment may alter multiple properties, consider an example that involves adding a salt to increase water phase salinity (WPS), which might be immediately successful at its overall aim (i.e., an increase in a fluid's WPS), however, it may also reduce a fluid's yield point (YP). In a traditional sense, this treatment would be classed as a success as it fulfilled the stated purpose but could, in fact, be an overall negative for the fluid system. As an example, a framework can implement a CI-based approach that enables a greater understanding of the status of the fluid before and after treatment and thus an improvement or otherwise to the fluid's quality can be assessed automatically. Furthermore, such an approach acts as an enabler for parametric fluid management, for example, where treatments can be made with the aim of overall fluid health rather than treatments being aimed at specific properties. Thus, improved and/or automated decisions on treatments can be made via a much greater understanding of the effects on the entire fluid.

As an example, a framework may provide for self-learning for assessing fluid treatment effectiveness. For example, a CI-based approach can be used to assess the effects on entire fluid treatments made to a system, which, in turn, can be utilized for self-learning of a framework. As an example, a framework can create a global condition index from automated measurements where a treatment is recommended based on individual CIs where resulting fluid properties can be measured. Such new measurements may be used to create a new global CI, which gives an understanding on the effect, positive or negative on a fluid system. In such an approach, each cycle may be added to a case history, which can be fed into a learning workflow.

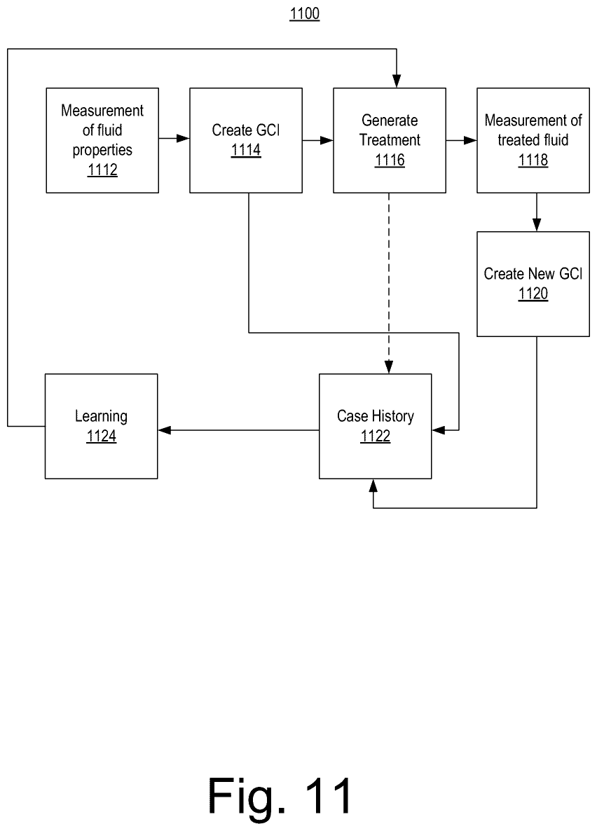

FIG. 11 shows an example of a workflow 1100 that includes a measurements block 1112 for measuring fluid properties, a creation block 1114 for creating a GCI based on measured fluid properties, a generation block 1116 for generating a treatment (e.g., advice, recommendation, control action, etc.) based at least in part on the GCI, a measurement block 1118 for measuring fluid properties during and/or after implementation of the treatment, a creation block 1120 for creating a new GCI based on the additional measured fluid properties, and a case history block 1122 for recording various GCI values along with one or more recommended treatments as implemented. As shown, a learning block 1124 can access the case history block 1122 for purposes of learning, for example, for training and/or re-training one or more machine learning models (ML models) that can be implemented for purposes of generating a treatment or treatments, for example, per the generation block 1116.

As an example, learning may modify one or more processes for generation of treatment recommendations, which may include modification of one or more treatments, based on how much a GCI has changed in the past, which may be utilized to prioritize one or more otherwise suitable treatment options. As an example, learning may be utilized to predict and/or otherwise model the most likely effects of a proposed treatment, which may allow an operator and/or controller to change an approach to treatment (e.g., a potential treatment) prior to addition of one or more additives. While additives are mentioned, fluid treatment may involve one or more processes such as a physical process (e.g., addition of a screener, a mixer, etc.).

As an example, a framework may generate a recommendation for optimal fluid rheology for ECD management and for tripping out of a hole (e.g., POOH). For example, a framework may utilize one or more hydraulic models to produce parametric simulations that indicate optimal conditions for a desired ROP, hole-cleaning situation or pump pressure. Use of such simulations may conventionally involve treating a fluid's rheology as static while varying one or more other parameters. As an example, a framework can extend use of simulations to treat fluid rheology as a variable, which, in effect may involve back-calculating target rheology (e.g., which may be given to an engineer). In an individual condition, parameters such as flow rate may be locked but in the overall simulation matrix, a range of outcomes may be simulated. In such an approach, a framework may indicate potential benefit to a chosen parameter, recommend the rheology target, which may be implemented via a treatment, be tested, etc. In such an approach, qualitative treatment advice for how to do this may be provided, for example, for implementation by machine, human, human and machine, etc.

As an example, a framework may utilize one or more simulations to determine a potential reduction in tripping time (e.g., RIH, POOH, etc.). For example, if a potential reduction is identified, then a recommendation may be generated that aims to achieve the potential reduction. In such an approach, a potential reduction in tripping time may be associated with a decrease in cost due to flat time while tripping (e.g., a reduction in NPT, etc.).

As an example, a framework may implement one or more simulators that can provide for simulating one or more drilling operations and/or one or more treatment effects. In such an approach, timings may be involved where timing of a treatment effect is weighed against timing of a drilling operation, particular where a change in state of a rig system may occur (e.g., from drilling to POOH). Where the framework determines that benefit from a treatment as to one state is likely to be short-lived due to a change in state and, for example, detrimental to a future state that is to occur within a relatively short period of time, the framework may provide an indication as to the treatment and leave it up to a human in the loop (HITL) to decide whether the treatment is to be implemented given the pros/cons generated by the framework.

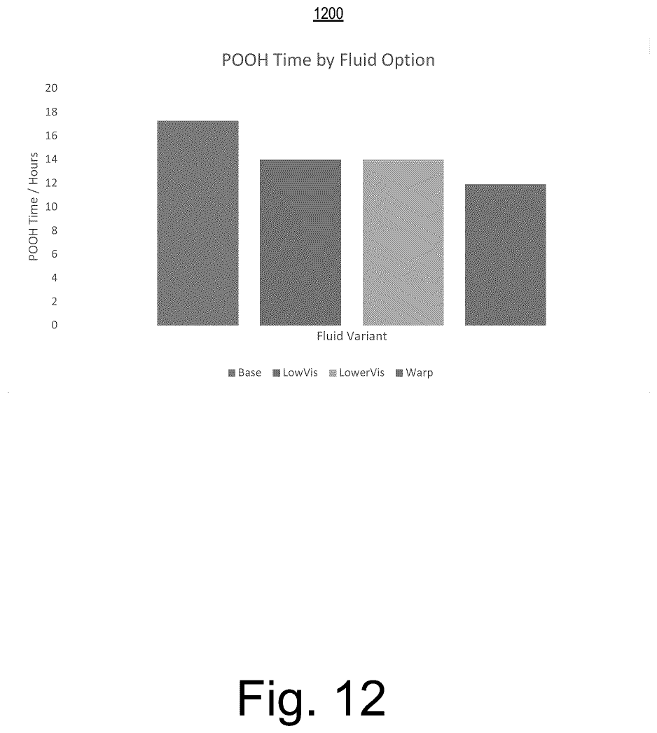

FIG. 12 shows an example of a plot 1200 of POOH time for various fluid options, which may be different treatment options. As indicated, a base option has the greatest POOH time while a warp option has the shortest POOH time. As an example, a treatment may be generated along with estimates of impact on one or more drilling operations, which may be current drilling operations and/or future drilling operations.

A US nonprovisional patent application having Ser. No. 18/295,359, filed 4 Apr. 2023, is incorporated by reference herein in its entirety. A US provisional patent application having Attorney Docket No. IS22.0842-US-PSP and Ser. No. 63/541484, filed 29 Sep. 2023, is incorporated by reference herein in its entirety.

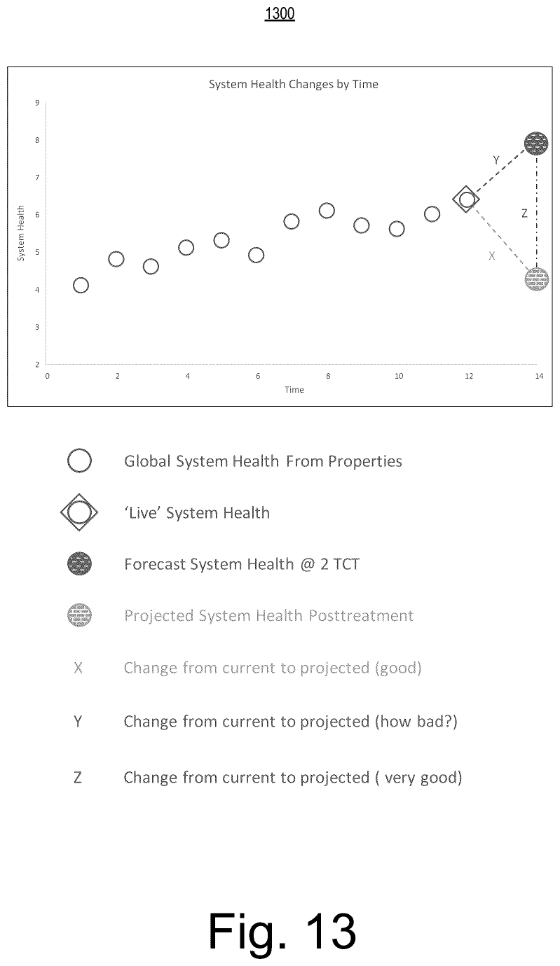

FIG. 13 shows an example plot 1300 of system health versus time where system health may be characterized using a CI-based approach and/or one or more other approaches. As shown, past indicators and a current “live” indicator can be presented (e.g., as a GUI) where forecast (e.g., without treatment) and projected (e.g., with treatment) can be presented, which may provide for an indication as to a difference, which may be quantitatively and/or qualitatively assessed. As explained, a GCI may be based on multiple property CIs and, for example, the plot 1300, as a GUI, may provide for interactivity such that a user may select a graphic such that one or more individual and/or customized indicators are generated and rendered (e.g., to inspect how individual property CIs may be improved, etc.).

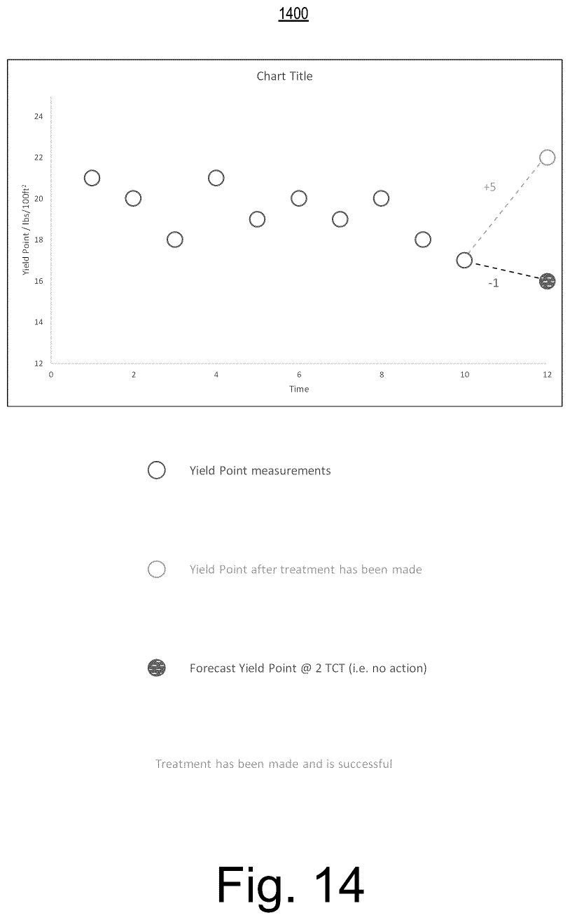

FIG. 14 shows an example of a plot 1400, which may be a GUI, for a particular property (e.g., YP) where an increase in the value of the property may be deemed beneficial. As shown, a forecast may be generated by a framework that indicates a decrease whereas a treatment recommended by the framework can provide for a projected increase (e.g., a predicted value of YP).

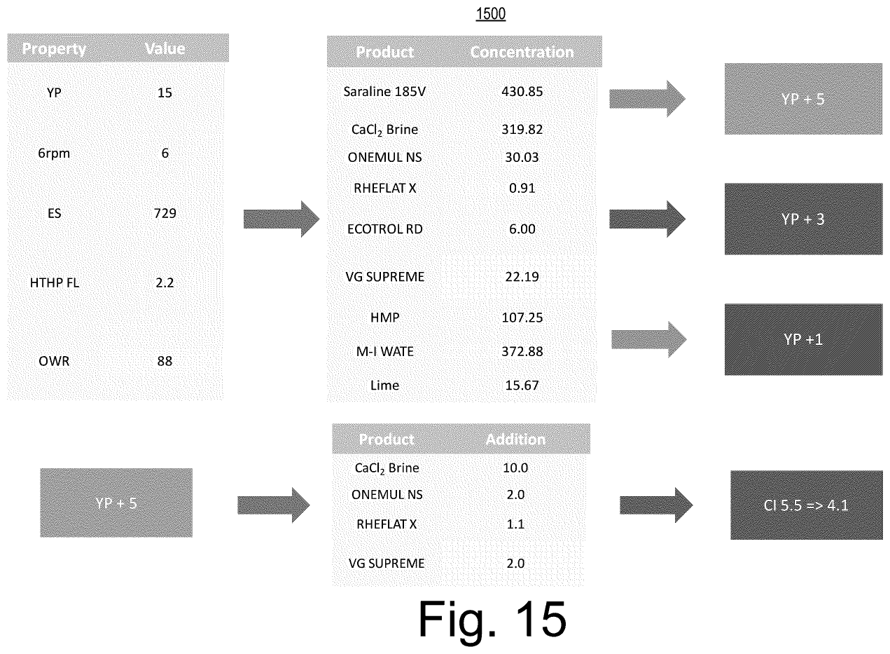

FIG. 15 shows an example of a GUI 1500 that can provide for generation of one or more treatments using one or more additives. As an example, a framework may provide for performing one or more inversions (e.g., model-based inversions, etc.), for example, to generate one or more recommended treatments. As shown, a recommendation may be linked to an outcome. For example, a framework can generate one or more recommendations and then generate a prediction as to an outcome for implementation thereof (e.g., a treatment involving additives, etc.). As shown, a CI approach may be utilized to characterize an outcome.

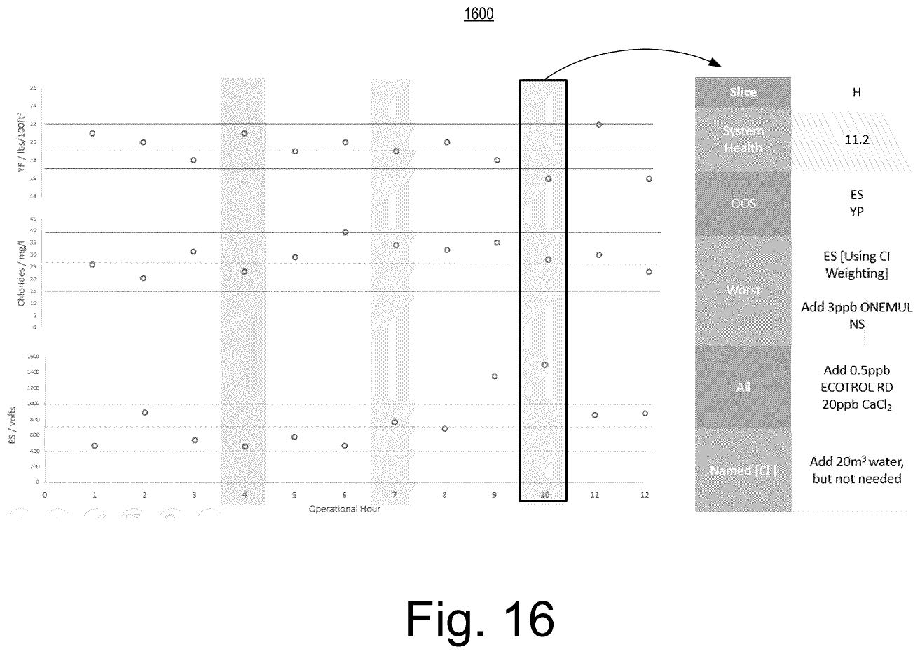

FIG. 16 shows an example of a GUI 1600 that can provide for monitoring of properties using one or more indicators where, for example, for a point in time (e.g., a time window, etc.), a framework may generate metrics that characterize a fluid system and may generate one or more recommendations as to achieving improved health of the fluid system.

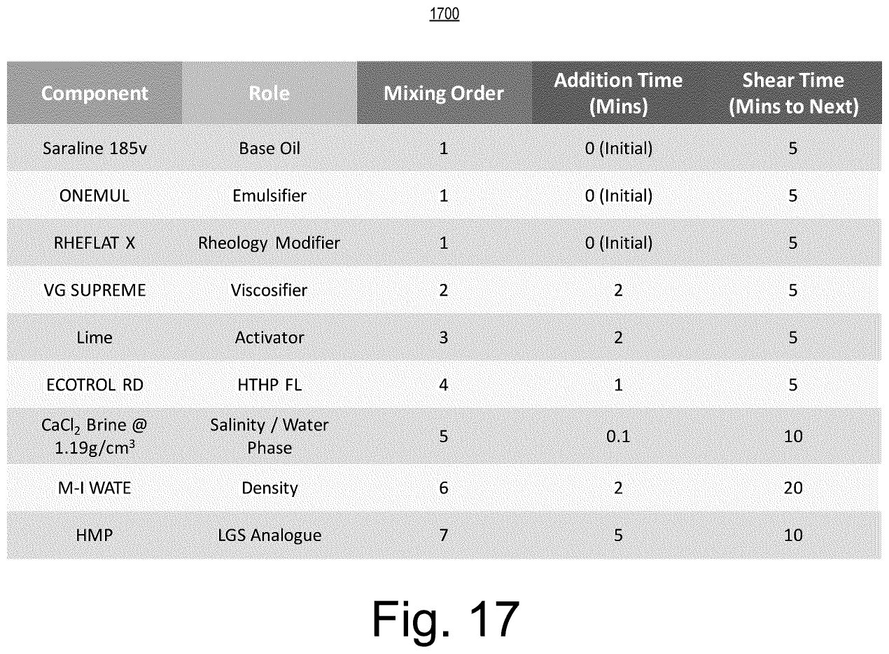

FIG. 17 shows an example of a GUI 1700 that can provide for describing one or more types of additives, which may be part of a formulation that may be mixed in a tank, pre-mixed, etc. As an example, such a GUI may provide for an order of mixing, mixing times, mixing equipment, mixing speed, etc. As explained, time may be a factor as to time to implement, which may depend on time to mix.

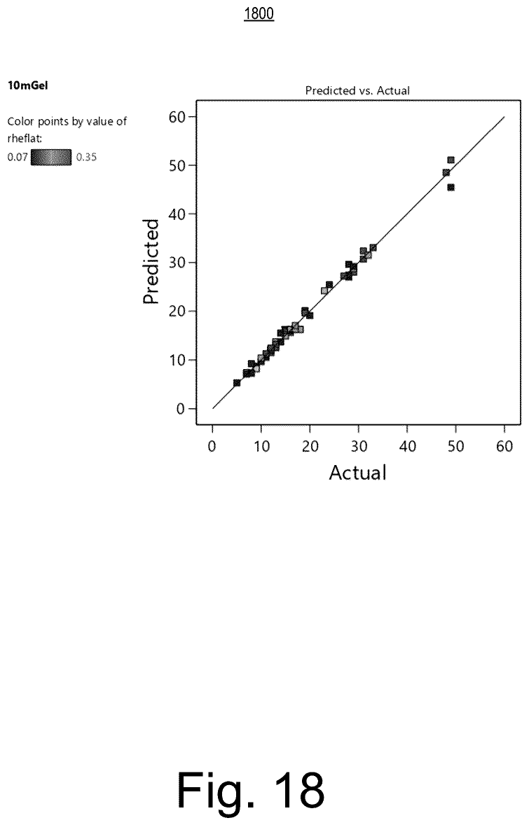

FIG. 18 shows an example of a plot 1800 of predicted versus actual property values for 10 minute gel using a model that may be part of a framework. In general, 10 m gel is a property that may be difficult to predict, due to it being the result of complex electrochemical interactions. As shown in FIG. 18, the 10 m gel is predicted very reliably by the model, which may consider low-gravity solids (LGS). LGS is a type of drilling-fluid solid having a lower density than the barite or hematite that may be used to weight up a drilling fluid, including drill solids plus the added bentonite clay. As an example, a fluid engineer may compute concentration of these and other types of solids on the basis of mud weight, retort analysis, chloride titrations and other information. Solids may be reported as Ibm/bbl or vol. %. Water is 1.0, barite 4.20, and hematite 5.505 g/cm3. Low-gravity solids are normally assumed to have a density of 2.60 g/cm3. As explained, a framework may take LGS into account and may recommend a treatment that may alter LGS.

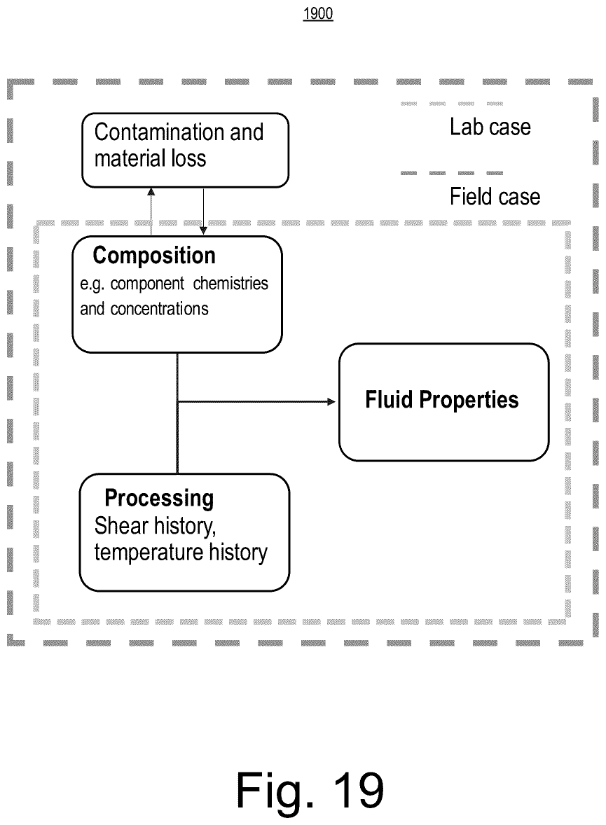

FIG. 19 shows an example of a model-building process 1900 that can consider composition and processing where composition may account for contamination and material loss. As shown, the process 1900 can utilize lab and/or field data.

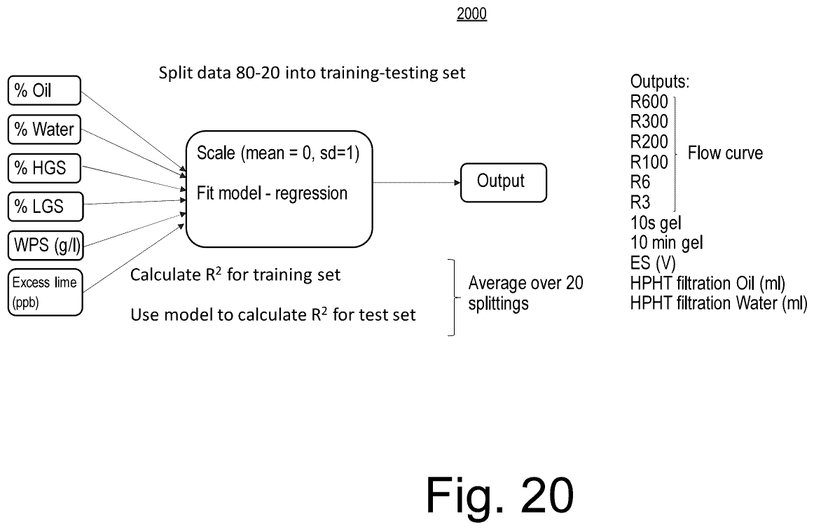

FIG. 20 shows an example of a model-building process 2000 that includes training a regression model. As shown, data may be split for purposes of training and testing.

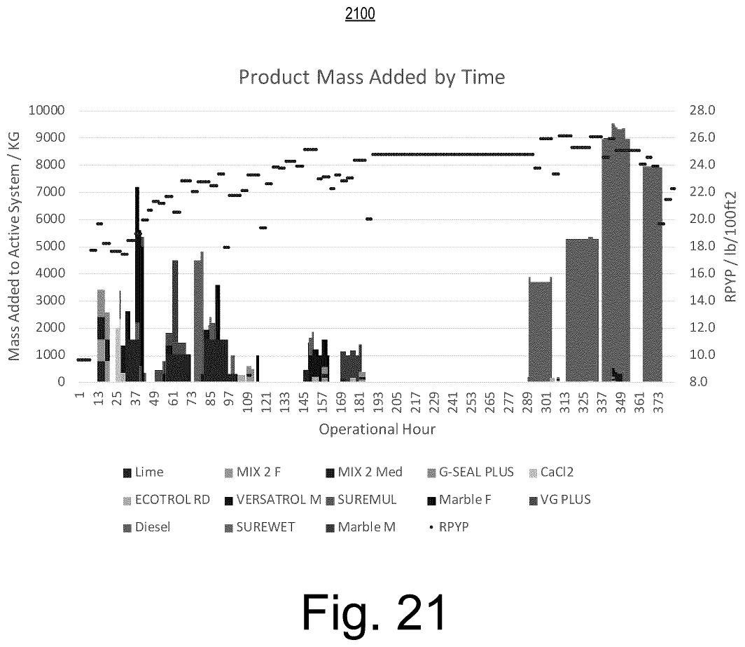

FIG. 21 shows an example GUI 2100 that includes various details of product mass added by time. As an example, such data may be stored in a database and accessed for purposes of training, re-training, etc. As explained, a framework may provide for self-learning, which may include self-learning based on such data.

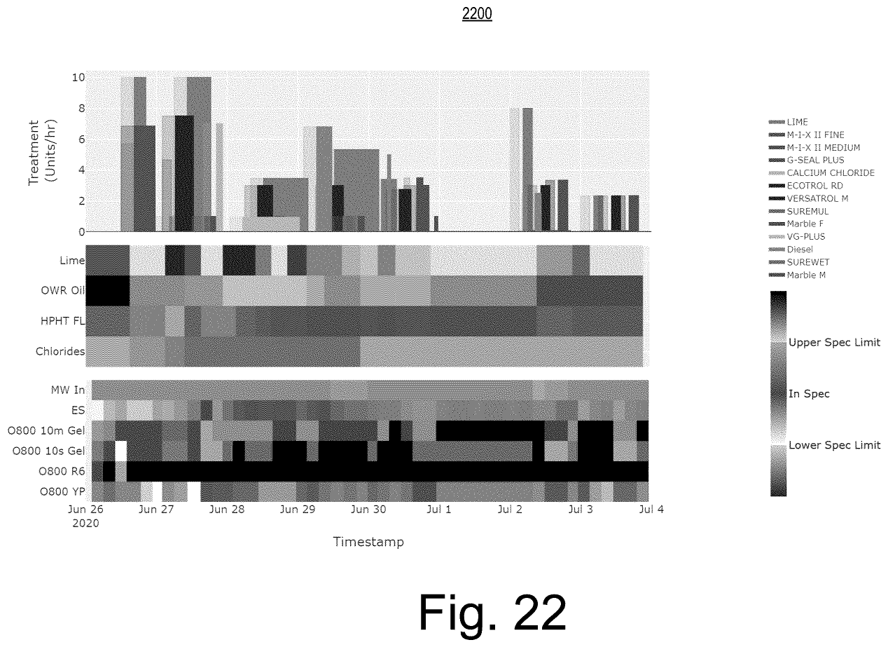

FIG. 22 shows an example GUI 2200 that can provide for visualization of various treatment information, along with property specifications (e.g., limits, targets, etc.), which may be rendered with respect to time.

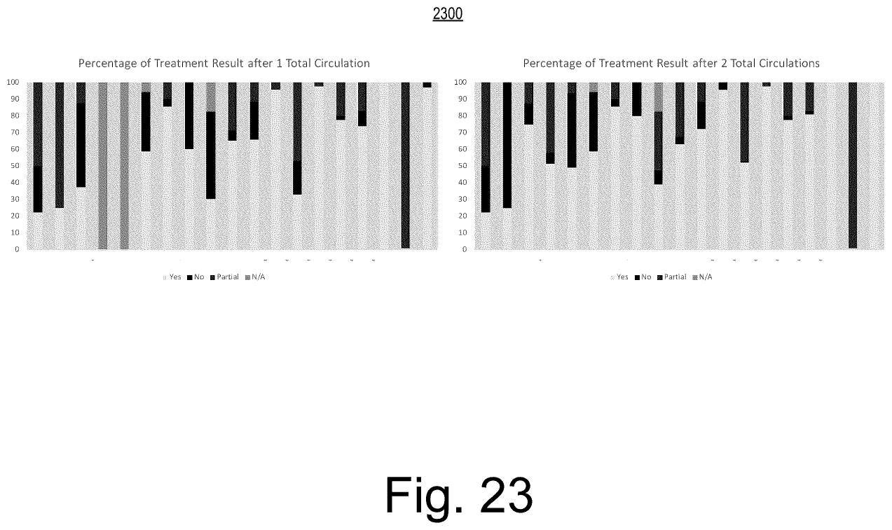

FIG. 23 shows an example GUI 2300 of success ratings with respect to circulation, specifically for 1 total circulation and 2 total circulations. As an example, a framework may account for how many circulations may be required to achieve a desired benefit, which may be related to flow rate, depth of hole, downhole volume, tank volume, etc.

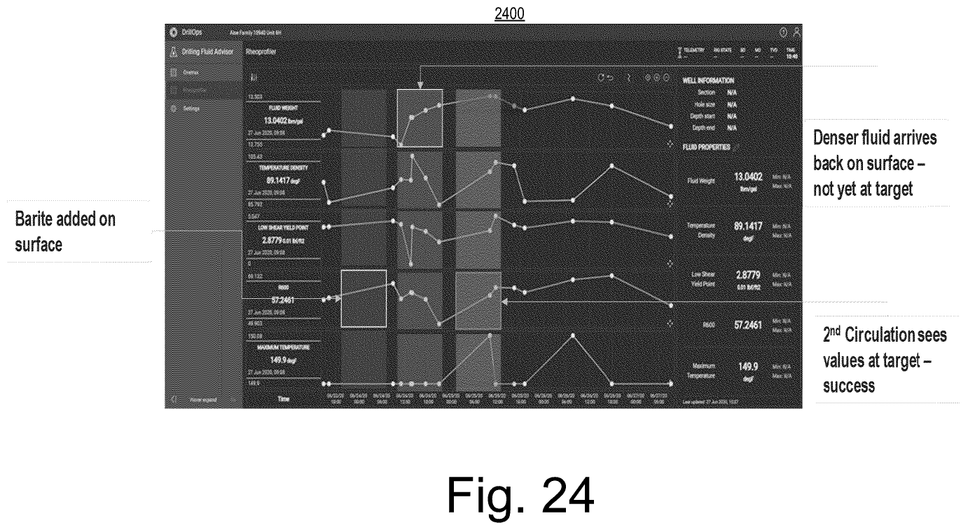

FIG. 24 shows an example GUI 2400 that can provide various types of information as to fluid system characteristics, which can include indicators for treatments, etc. In such an example, when a product is added, a framework can create a unique record, linked to the treatment objective, which may then be compared to the objective, target property and addition status after 1, then 2 circulations with an assessment of its success. As an example, the GUI 2400 may indicate an initial addition of barite as being detected by the framework where the framework identifies this as treating with a density sub-state. As shown, a period of circulation may be identified as conditioning with a return to drilling once the density is sufficiently even.

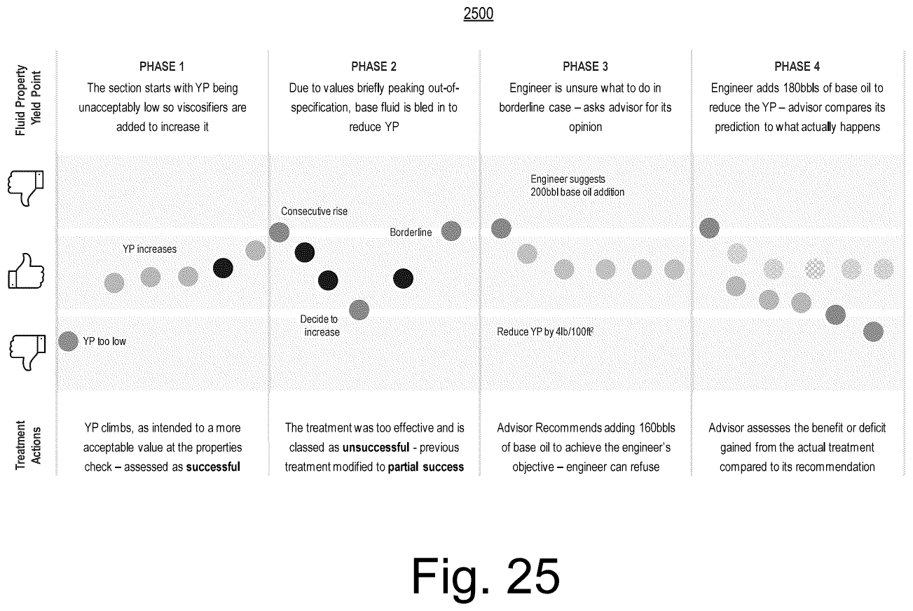

FIG. 25 shows an example of a GUI 2500 that can correspond to a workflow where, for example, a human may utilize a framework for guidance. As shown, the framework may provide for various assessments and may perform self-learning to improve its operation.

As explained, one or more types of condition indexes may be utilized. As an example, for inversion, a framework may determine one or more set of additions that will bring one or more parameters (e.g., rheological and/or other) as close as possible to a design specification (e.g., target, etc.). As an example, a workflow may have involved drilling and thereby accumulating some LGS material into a drilling fluid. In such an example, a framework may determine what one or more further additions may be made to as close as possible return fluid properties to that of a target specification. In such an example, a framework may provide for inverting a set of equations; however, in various instances such an approach may not be practicable, particularly when constraints on each of the responses are included and/or where real-time answers are desired. As an example, a framework may implement one or more statistical types of approaches such as, for example, a Monte-Carlo method. As an example, a statistical approach may include defining a domain of possible inputs, generating inputs randomly from a probability distribution over the domain, performing a deterministic computation on the inputs, and aggregating results.

As an example, a framework may choose a random set of values (e.g., of formulation parameters) and compute resulting measurement parameters using a model. In such an example, values may then be adjusted to follow a steepest descent algorithm to find an optimal solution against one or more provided target parameters. If a multi-dimensional equation is relatively simple, such an approach may be practical. However, various systems of equations may include multiple local minima. Hence, for global optimization, many different starting points may be tried by a framework such that a multi-dimensional space is sufficiently explored, and chances of finding the desired global optimum increased.

FIG. 26 shows various example equations and a plot 2600. In such an approach, goodness of a final fit may be described by a “Desirability” parameter defined by the first two equations in FIG. 26 where, for example, rᵢ are “Importance” values which may vary from 1 to 5. In such an approach, each dᵢ can be a desirability of an individual parameter, for example, consider the equations for dᵢ in FIG. 26. In FIG. 26, a plot is shown as a design-expert “desireability” function shape.

FIG. 27 shows various example equations and a plot 2700 as to an example condition index and an alternative example condition index modified as a cost function.

As to a Pricilla's condition index equation set, consider the first set of equations in FIG. 27. With such an approach, a curve may be continuous, but with a slope jump at the limits Lowᵢ and Highᵢ. As to the example alternative condition index modified as cost function, consider that at Highᵢ the parameter either side is the same and also that the slope is the same. However, instead of trying to match the condition index across the boundary, a framework may define a new function that is numerically very close to the condition index, as illustrated in FIG. 27 by the second set of equations. In such an example, with wtᵢ=1, this function closely approximates Priscilla's corrected function (see first set of equations). As an example, the parameter wtᵢ may be changed on either side such that the function becomes asymmetric or Lowᵢ and Highᵢ can be adjusted. The effect of wtᵢ is similar to that shown in the plot of FIG. 27. As an example, an appropriate cost function may be presented as the third equation in FIG. 27, as given for dᵢ. As to the plot of FIG. 27, it corresponds to Ti=50, Highᵢ=55, Lowᵢ=45, and wtᵢ=2. The cost function given in FIG. 27 as to dᵢ may be combined with one or more other parameter cost functions, for example, as in the first two equations of FIG. 26, which may provide for a combined cost function.

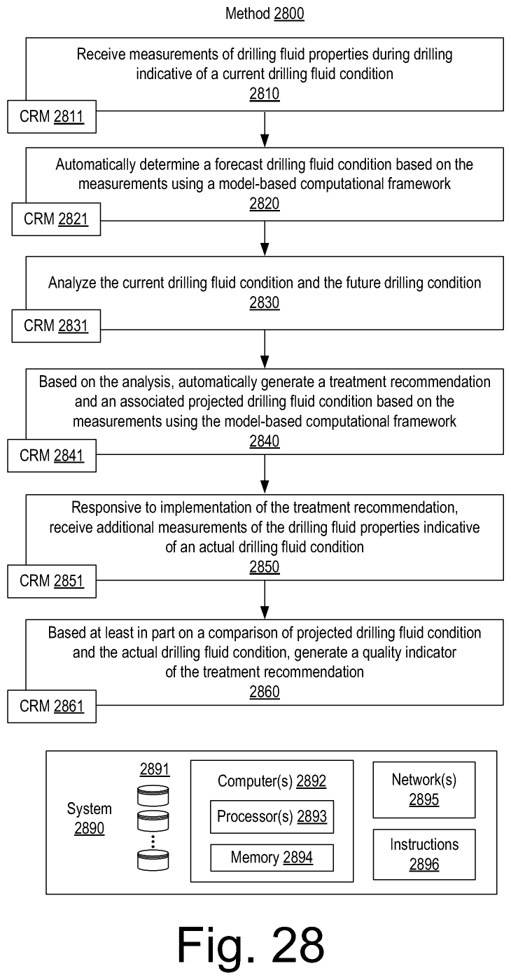

FIG. 28 shows an example of a method 2800 and an example of a system 2890. As shown, the method 2800 may include a reception block 2810 for receiving measurements of drilling fluid properties of drilling fluid during drilling indicative of a current drilling fluid condition; a determination block 2820 for automatically determining a forecast drilling fluid condition based on the measurements using a model-based computational framework; an analysis block 2830 for analyzing the current drilling fluid condition and the future drilling condition; a generation block 2840 for, based on the analyzing, automatically generating a treatment recommendation and an associated projected drilling fluid condition based on the measurements using the model-based computational framework; a reception block 2850 for, responsive to implementation of the treatment recommendation, receiving additional measurements of the drilling fluid properties indicative of an actual drilling fluid condition; and a generation block 2860 for, based at least in part on a comparison of projected drilling fluid condition and the actual drilling fluid condition, generating a quality indicator of the treatment recommendation.

FIG. 28 also shows various computer-readable media (CRM) blocks 2811, 2821, 2831, 2841, 2851 and 2861. Such blocks may include instructions that are executable by one or more processors, which may be one or more processors of a computational framework, a system, a computer, etc. A computer-readable medium may be a computer-readable storage medium that is not a signal, not a carrier wave and that is non-transitory. For example, a computer-readable medium may be a physical memory component that may store information in a digital format.

In the example of FIG. 28, a system 2890 includes one or more information storage devices 2891, one or more computers 2892, one or more networks 2895 and instructions 2896. As to the one or more computers 2892, each computer may include one or more processors (e.g., or processing cores) 2893 and memory 2894 for storing the instructions 2896, for example, executable by at least one of the one or more processors. As an example, a computer may include one or more network interfaces (e.g., wired or wireless), one or more graphics cards, a display interface (e.g., wired or wireless), etc. The system 2890 may be specially configured to perform one or more portions of the method 2800 of FIG. 28.

As an example, a computational framework may include a solver, which may be implemented via executable instructions. For example, consider a computational framework that includes a processor and memory accessible to the processor where executable instructions may be stored in the memory and accessed for execution by the processor to cause the computational framework to perform one or more actions. Such a computational framework may include one or more interfaces for receipt of information and/or for output of information, which may include values of parameters, an instruction, etc. As an example, a computational framework may be part of a controller. As an example, a computational framework may be part of a system.

As an example, various systems, methods, etc., may implement one or more ML models. As to types of ML models, consider one or more of a support vector machine (SVM) model, a k-nearest neighbors (KNN) model, an ensemble classifier model, a neural network (NN) model, etc. As an example, a machine learning model may be a deep learning model (e.g., deep Boltzmann machine, deep belief network, convolutional neural network, stacked auto-encoder, etc.), an ensemble model (e.g., random forest, gradient boosting machine, bootstrapped aggregation, AdaBoost, stacked generalization, gradient boosted regression tree, etc.), a neural network model (e.g., radial basis function network, perceptron, back-propagation, Hopfield network, etc.), a regularization model (e.g., ridge regression, least absolute shrinkage and selection operator, elastic net, least angle regression), a rule system model (e.g., cubist, one rule, zero rule, repeated incremental pruning to produce error reduction), a regression model (e.g., linear regression, ordinary least squares regression, stepwise regression, multivariate adaptive regression splines, locally estimated scatterplot smoothing, logistic regression, etc.), a Bayesian model (e.g., naïve Bayes, average on-dependence estimators, Bayesian belief network, Gaussian naïve Bayes, multinomial naïve Bayes, Bayesian network), a decision tree model (e.g., classification and regression tree, iterative dichotomiser 3, C4.5, C5.0, chi-squared automatic interaction detection, decision stump, conditional decision tree, M5), a dimensionality reduction model (e.g., principal component analysis, partial least squares regression, Sammon mapping, multidimensional scaling, projection pursuit, principal component regression, partial least squares discriminant analysis, mixture discriminant analysis, quadratic discriminant analysis, regularized discriminant analysis, flexible discriminant analysis, linear discriminant analysis, etc.), an instance model (e.g., k-nearest neighbor, learning vector quantization, self-organizing map, locally weighted learning, etc.), a clustering model (e.g., k-means, k-medians, expectation maximization, hierarchical clustering, etc.), etc.

As an example, a system may utilize one or more recurrent neural networks (RNNs). One type of RNN is referred to as long short-term memory (LSTM), which may be a unit or component (e.g., of one or more units) that may be in a layer or layers. A LSTM component may be a type of artificial neural network (ANN) designed to recognize patterns in sequences of data, such as time series data. When provided with time series data, LSTMs take time and sequence into account such that an LSTM may include a temporal dimension. For example, consider utilization of one or more RNNs for processing temporal data from one or more sources, optionally in combination with spatial data. Such an approach may recognize temporal patterns, which may be utilized for making predictions (e.g., as to a pattern or patterns for future times, etc.).

As an example, the TENSORFLOW framework (Google LLC, Mountain View, California) may be implemented, which is an open-source software library for dataflow programming that includes a symbolic math library, which may be implemented for machine learning applications that may include neural networks. As an example, the CAFFE framework may be implemented, which is a DL framework developed by Berkeley AI Research (BAIR) (University of California, Berkeley, California). As another example, consider the SCIKIT platform (e.g., scikit-learn), which utilizes the PYTHON programming language. As an example, a framework such as the APOLLO AI framework may be utilized (APOLLO.AI GmbH, Germany). As mentioned, a framework such as the PYTORCH framework may be utilized.

As an example, a training method may include various actions that may operate on a dataset to train a ML model. As an example, a dataset may be split into training data and test data where test data may provide for evaluation. A method may include cross-validation of parameters and best parameters, which may be provided for model training.

The TENSORFLOW framework may run on multiple CPUs and GPUs (with optional CUDA (NVIDIA Corp., Santa Clara, California) and SYCL (The Khronos Group Inc., Beaverton, Oregon) extensions for general-purpose computing on graphics processing units (GPUs)). TENSORFLOW is available on 64-bit LINUX, MACOS (Apple Inc., Cupertino, California), WINDOWS (Microsoft Corp., Redmond, Washington), and mobile computing platforms including ANDROID (Google LLC, Mountain View, California) and IOS (Apple Inc.) operating system-based platforms.

TENSORFLOW computations may be expressed as stateful dataflow graphs; noting that the name TENSORFLOW derives from the operations that such neural networks perform on multidimensional data arrays. Such arrays may be referred to as “tensors”.

As an example, a method can include receiving measurements of drilling fluid properties of drilling fluid during drilling indicative of a current drilling fluid condition; automatically determining a forecast drilling fluid condition based on the measurements using a model-based computational framework; analyzing the current drilling fluid condition and the future drilling condition; based on the analyzing, automatically generating a treatment recommendation and an associated projected drilling fluid condition based on the measurements using the model-based computational framework; responsive to implementation of the treatment recommendation, receiving additional measurements of the drilling fluid properties indicative of an actual drilling fluid condition; and, based at least in part on a comparison of projected drilling fluid condition and the actual drilling fluid condition, generating a quality indicator of the treatment recommendation.

As an example, drilling fluid properties can include one or more of yield point, electric stability, density and gel test.

As an example, automatically determining a forecast drilling fluid condition can include computing one or more condition indexes and/or computing a global condition index. In such an example, a global condition index may depend on multiple condition indexes.

As an example, automatically generating a treatment recommendation may include selecting the treatment recommendation from a plurality of different treatment recommendations.

As an example, a treatment recommendation can include a recommendation for addition of one or more materials to the drilling fluid. In such an example, at least one of the one or more materials may include barite.

As an example, a treatment recommendation can include a recommendation for addition of one or more of a base oil, an emulsifier, a rheology modifier, a viscosifier, an activator, a brine, a density modifier and a low-gravity solids analogue.

As an example, a treatment recommendation can include a recommended order for addition material.

As an example, a method can include receiving additional measurements of drilling fluid properties indicative of an actual drilling fluid condition in a manner that includes receiving additional measurements after at least one circulation cycle.

As an example, a model-based computational framework may utilize at least one regression model. As an example, a model-based computational framework may utilize at least one machine learning model.

As an example, a method can include generating a graphical user interface of additives added to drilling fluid with respect to time. In such an example, the additives may include at least one additive associated with a treatment recommendation.

As an example, a method can include tracking mass of additives added to drilling fluid with respect to time.

As an example, a method can include generating a graphical user interface of drilling fluid properties with respect to time coded using one or more limits (e.g., an upper specification limit, a lower specification limit, etc.). In such an example, the graphical user interface may include a visualization of treatments applied to the drilling fluid with respect to time (e.g., in units per hour, etc.).

As an example, a system can include one or more processors; memory accessible to at least one of the one or more processors; processor-executable instructions stored in the memory and executable to instruct the system to: receive measurements of drilling fluid properties of drilling fluid during drilling indicative of a current drilling fluid condition; automatically determine a forecast drilling fluid condition based on the measurements using a model-based computational framework; analyze the current drilling fluid condition and the future drilling condition; based on the analysis, automatically generate a treatment recommendation and an associated projected drilling fluid condition based on the measurements using the model-based computational framework; responsive to implementation of the treatment recommendation, receive additional measurements of the drilling fluid properties indicative of an actual drilling fluid condition; and, based at least in part on a comparison of projected drilling fluid condition and the actual drilling fluid condition, generate a quality indicator of the treatment recommendation.

As an example, one or more computer-readable storage media can include processor-executable instructions to instruct a computing system to: receive measurements of drilling fluid properties of drilling fluid during drilling indicative of a current drilling fluid condition; automatically determine a forecast drilling fluid condition based on the measurements using a model-based computational framework; analyze the current drilling fluid condition and the future drilling condition; based on the analysis, automatically generate a treatment recommendation and an associated projected drilling fluid condition based on the measurements using the model-based computational framework; responsive to implementation of the treatment recommendation, receive additional measurements of the drilling fluid properties indicative of an actual drilling fluid condition; and, based at least in part on a comparison of projected drilling fluid condition and the actual drilling fluid condition, generate a quality indicator of the treatment recommendation.

As an example, a computer program product that may include computer-executable instructions to instruct a computing system to perform one or more methods such as one or more of the methods described herein (e.g., in part, in whole and/or in various combinations).

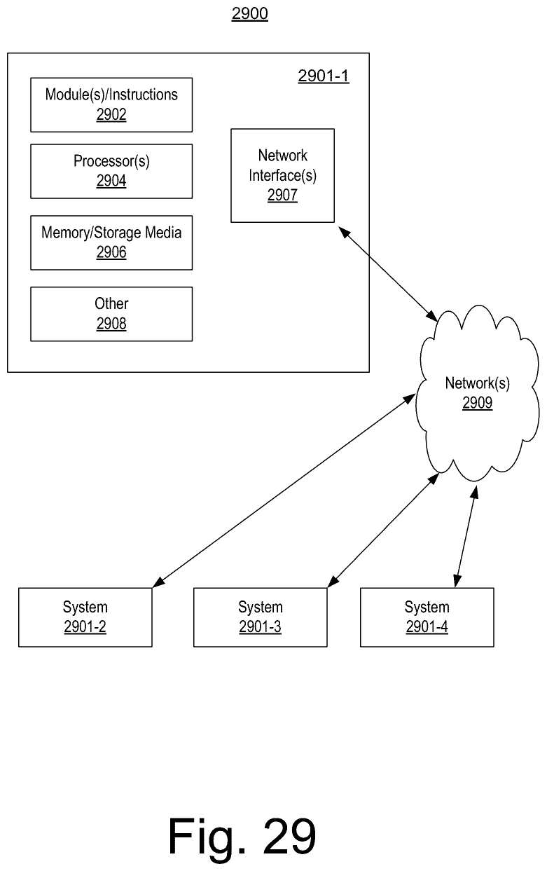

In some embodiments, a method or methods may be executed by a computing system. FIG. 29 shows an example of a system 2900 that may include one or more computing systems 2901-1, 2901-2, 2901-3 and 2901-4, which may be operatively coupled via one or more networks 2909, which may include wired and/or wireless networks. As shown, the system 2900 may include one or more other components 2908.

As an example, a system may include an individual computer system or an arrangement of distributed computer systems. In the example of FIG. 29, the computer system 2901-1 may include one or more modules 2902, which may be or include processor-executable instructions, for example, executable to perform various tasks (e.g., receiving information, requesting information, processing information, simulation, outputting information, etc.).

As an example, a module may be executed independently, or in coordination with, one or more processors 2904, which is (or are) operatively coupled to one or more storage media 2906 (e.g., via wire, wirelessly, etc.). As an example, one or more of the one or more processors 2904 may be operatively coupled to at least one of one or more network interface 2907. In such an example, the computer system 2901-1 may transmit and/or receive information, for example, via the one or more networks 2909 (e.g., consider one or more of the Internet, a private network, a cellular network, a satellite network, etc.). As shown, one or more other components 2908 may be included in the computer system 2901-1.

As an example, the computer system 2901-1 may receive from and/or transmit information to one or more other devices, which may be or include, for example, one or more of the computer systems 2901-2, etc. A device may be located in a physical location that differs from that of the computer system 2901-1. As an example, a location may be, for example, a processing facility location, a data center location (e.g., server farm, etc.), a rig location, a wellsite location, a downhole location, etc.

As an example, a processor may be or include a microprocessor, microcontroller, processor module or subsystem, programmable integrated circuit, programmable gate array, or another control or computing device.

As an example, the storage media 2906 may be implemented as one or more computer-readable or machine-readable storage media. As an example, storage may be distributed within and/or across multiple internal and/or external enclosures of a computing system and/or additional computing systems.

As an example, a storage medium or storage media may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLUERAY disks, or other types of optical storage, or other types of storage devices.

As an example, a storage medium or media may be located in a machine running machine-readable instructions, or located at a remote site from which machine-readable instructions may be downloaded over a network for execution.

As an example, various components of a system such as, for example, a computer system, may be implemented in hardware, software, or a combination of both hardware and software (e.g., including firmware), including one or more signal processing and/or application specific integrated circuits.

As an example, a system may include a processing apparatus that may be or include a general-purpose processors or application specific chips (e.g., or chipsets), such as ASICs, FPGAs, PLDs, or other appropriate devices.

As an example, a device may be a mobile device that includes one or more network interfaces for communication of information. For example, a mobile device may include a wireless network interface (e.g., operable via IEEE 802.11, ETSI GSM, BLUETOOTH, satellite, etc.). As an example, a mobile device may include components such as a main processor, memory, a display, display graphics circuitry (e.g., optionally including touch and gesture circuitry), a SIM slot, audio/video circuitry, motion processing circuitry (e.g., accelerometer, gyroscope), wireless LAN circuitry, smart card circuitry, transmitter circuitry, GPS circuitry, and a battery. As an example, a mobile device may be configured as a cell phone, a tablet, etc. As an example, a method may be implemented (e.g., wholly or in part) using a mobile device. As an example, a system may include one or more mobile devices.

As an example, a system may be a distributed environment, for example, a so-called “cloud” environment where various devices, components, etc. interact for purposes of data storage, communications, computing, etc. As an example, a device or a system may include one or more components for communication of information via one or more of the Internet (e.g., where communication occurs via one or more Internet protocols), a cellular network, a satellite network, etc. As an example, a method may be implemented in a distributed environment (e.g., wholly or in part as a cloud-based service).

As an example, information may be input from a display (e.g., consider a touchscreen), output to a display or both. As an example, information may be output to a projector, a laser device, a printer, etc. such that the information may be viewed. As an example, information may be output stereographically or holographically. As to a printer, consider a 2D or a 3D printer. As an example, a 3D printer may include one or more substances that may be output to construct a 3D object. For example, data may be provided to a 3D printer to construct a 3D representation of a subterranean formation. As an example, layers may be constructed in 3D (e.g., horizons, etc.), geobodies constructed in 3D, etc. As an example, holes, fractures, etc., may be constructed in 3D (e.g., as positive structures, as negative structures, etc.).

Although only a few examples have been described in detail above, those skilled in the art will readily appreciate that many modifications are possible in the examples. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

What is claimed is:

1. A method comprising:
receiving measurements of drilling fluid properties of drilling fluid during drilling indicative of a current drilling fluid condition;
automatically determining a forecast drilling fluid condition based on the measurements using a model-based computational framework;
analyzing the current drilling fluid condition and the future drilling condition;
based on the analyzing, automatically generating a treatment recommendation and an associated projected drilling fluid condition based on the measurements using the model-based computational framework;
responsive to implementation of the treatment recommendation, receiving additional measurements of the drilling fluid properties indicative of an actual drilling fluid condition; and
based at least in part on a comparison of projected drilling fluid condition and the actual drilling fluid condition, generating a quality indicator of the treatment recommendation.

2. The method of claim 1, wherein the drilling fluid properties comprise one or more of yield point, electric stability, density and gel test.

3. The method of claim 1, wherein the automatically determining comprises computing one or more condition indexes.

4. The method of claim 1, wherein the automatically determining comprises computing a global condition index.

5. The method of claim 4, wherein the global condition index depends on multiple condition indexes.

6. The method of claim 1, wherein the automatically generating the treatment recommendation comprises selecting the treatment recommendation from a plurality of different treatment recommendations.

7. The method of claim 1, wherein the treatment recommendation comprises a recommendation for addition of one or more materials to the drilling fluid.

8. The method of claim 7, wherein at least one of the one or more materials comprises barite.

9. The method of claim 1, wherein the treatment recommendation comprises a recommendation for addition of one or more of a base oil, an emulsifier, a rheology modifier, a viscosifier, an activator, a brine, a density modifier and a low-gravity solids analogue.

10. The method of claim 1, wherein the treatment recommendation comprises a recommended order for additional material.

11. The method of claim 1, wherein the receiving additional measurements of the drilling fluid properties indicative of an actual drilling fluid condition comprises receiving additional measurements after at least one circulation cycle.

12. The method of claim 1, wherein the model-based computational framework utilizes at least one regression model.

13. The method of claim 1, wherein the model-based computational framework utilizes at least one machine learning model.

14. The method of claim 1, comprising generating a graphical user interface of additives added to the drilling fluid with respect to time.

15. The method of claim 14, wherein the additives comprise at least one additive associated with the treatment recommendation.

16. The method of claim 1, comprising tracking mass of additives added to the drilling fluid with respect to time.

17. The method of claim 1, comprising generating a graphical user interface of the drilling fluid properties with respect to time coded using one or more limits.

18. The method of claim 17, wherein the graphical user interface further comprises a visualization of treatments applied to the drilling fluid with respect to time.

19. A system comprising:
one or more processors;
memory accessible to at least one of the one or more processors;
processor-executable instructions stored in the memory and executable to instruct the system to:
receive measurements of drilling fluid properties of drilling fluid during drilling indicative of a current drilling fluid condition;
automatically determine a forecast drilling fluid condition based on the measurements using a model-based computational framework;
analyze the current drilling fluid condition and the future drilling condition;
based on the analysis, automatically generate a treatment recommendation and an associated projected drilling fluid condition based on the measurements using the model-based computational framework;
responsive to implementation of the treatment recommendation, receive additional measurements of the drilling fluid properties indicative of an actual drilling fluid condition; and
based at least in part on a comparison of projected drilling fluid condition and the actual drilling fluid condition, generate a quality indicator of the treatment recommendation.

20. One or more computer-readable storage media comprising processor-executable instructions to instruct a computing system to:
receive measurements of drilling fluid properties of drilling fluid during drilling indicative of a current drilling fluid condition;
automatically determine a forecast drilling fluid condition based on the measurements using a model-based computational framework;
analyze the current drilling fluid condition and the future drilling condition;
based on the analysis, automatically generate a treatment recommendation and an associated projected drilling fluid condition based on the measurements using the model-based computational framework;
responsive to implementation of the treatment recommendation, receive additional measurements of the drilling fluid properties indicative of an actual drilling fluid condition; and
based at least in part on a comparison of projected drilling fluid condition and the actual drilling fluid condition, generate a quality indicator of the treatment recommendation.
